(19) United States
(12) Patent Application Publication
(10) Pub. No.: US 2026/0263429 A1
(43) Pub. Date: Sep. 10, 2026

(54) INHIBITORS OF CA 2+/CALMODULIN-DEPENDENT PROTEIN KINASE II (CAMKII) THAT DO NOT INHIBIT LONG-TERM POTENTIATION (LTP) INDUCTION AND THERAPEUTIC USES THEREOF

(71) Applicant: The Regents of the University of Colorado, A Body Corporate, Denver, CO (US)

(72) Inventors: K. Ulrich BAYER, Denver, CO (US); Matthew LARSEN, Denver, CO (US); Jonathan TULLIS, Denver, CO (US); Nicole RUMIAN, Denver, CO (US); Carolyn BROWN, Broomfield, CO (US); Steven COULTRAP, Aurora, CO (US)

(73) Assignee: The Regents of the University of Colorado, A Body Corporate, Denver, CO (US)

(21) Appl. No.: 19/167,319

(22) PCT Filed: Mar. 22, 2024
(86) PCT No.: PCT/US2024/021184

§ 371 Date: Sep. 19, 2025

Related U.S. Application Data
(60) Provisional application No. 63/454,007, filed on Mar. 22, 2023.
(60) Provisional application No. 63/547,087, filed on Nov. 2, 2023.

(51) Int. Cl. A61K 31/437; A61K 31/10; A61K 31/155; A61K 31/202; A61K 31/352; A61K 31/375; A61K 31/496; A61K 31/505; A61K 31/519; A61K 31/5377; A61K 39/395; A61P 25/28

(52) U.S. Cl. CPC A61K 31/437; A61K 31/10; A61K 31/155; A61K 31/202; A61K 31/352; A61K 31/375; A61K 31/496; A61K 31/505; A61K 31/519; A61K 31/5377; A61K 39/3955; A61P 25/28

(57) ABSTRACT

Embodiments of the instant disclosure relate to compositions and methods of making and using ATP-competitive Ca²⁺/Calmodulin-Dependent Protein Kinase II (CaMKII) inhibitors for treating a health condition. In certain embodiments, the health condition includes, but is not limited to, a health condition concerning the brain, and targeting the brain with one or more ATP-competitive CaMKII inhibitor to prevent, ameliorate, or treat the health condition. In some embodiments, ATP competitive CaMKII inhibitors of use herein preserve, restore, or induce long-term potentiation (LTP) mechanisms in the subject. In some embodiments, the health condition includes a chronic health condition, and the treatment includes a prolonged treatment of a subject having such a chronic health condition.

### PRIORITY

This International Application claims priority to U.S. Provisional Application No. 63/454,007 filed Mar. 22, 2023, and U.S. Provisional Application No. 63/547,087 filed Nov. 2, 2023. These applications are incorporated herein by reference in their entireties for all purposes.

### GOVERNMENT SUPPORT

This invention was made with government support under T32 grant number GM007635, F31 grant numbers AG069458, AG084197, and NS129254; and R01 grant numbers NS110383, NS081248, NS118786 and AG067713 awarded by the National Institutes of Health. The government has certain rights in this invention.

### FIELD

Embodiments of the instant disclosure relate to compositions and methods for using ATP competitive Ca²⁺/Calmodulin-Dependent Protein Kinase II (CaMKII) inhibitors in treating health conditions. In certain embodiments, the health condition includes, but is not limited to, a health condition concerning the brain and targeting the brain with one or more ATP competitive CaMKII inhibitors to prevent, ameliorate, or treat the health condition of the brain of a subject. In some embodiments, ATP competitive CaMKII inhibitors of use herein preserve or restore long-term potentiation (LTP) in a subject to improve brain function for short-term or prolonged use.

### BACKGROUND

Forms of synaptic plasticity, such as long-term potentiation (LTP) and long-term depression (LTD), contribute to learning, memory, and cognition. These brain functions are impaired in subjects having Alzheimer's disease (AD). One of the major pathological agents of AD, oligomeric amyloid-β peptide (Aβ), impairs hippocampal LTP. Aβ is produced by proteolytic cleavage of the Aβ precursor protein (APP). It has been suggested that APP acts upstream of Aβ (as the Aβ precursor) and downstream (as an Aβ effector). APP was found to be needed for the Aβ-induced LTP impairment. APP expression also increases after ischemic and traumatic brain injury. Additionally, the APP gene is in triplicate in Down Syndrome (DS) and the expression of APP and Aβ has been identified as increased under these condition. Further, LTP has been demonstrated to be impaired in both AD and DS, as well as, after models and remodeling of global cerebral ischemia, stroke, and traumatic brain injury (TBI).

Therefore, a need exists to identify additional therapeutic agents to treat these conditions having reduced side effects and where these therapeutic agents can be used for prolonged duration; for example, to treat chronic health conditions in an affected subject.

### SUMMARY

Embodiments of the instant disclosure relate to compositions and methods for using ATP competitive Ca²⁺/Calmodulin-Dependent Protein Kinase II (CaMKII) inhibitors in treating a health condition. In certain embodiments, the health condition includes, but is not limited to, a health condition concerning the brain and targeting the brain with one or more ATP competitive CaMKII inhibitors to prevent, ameliorate, or treat the health condition of the brain of a subject. In some embodiments, CaMKII inhibitors of use herein include, but are not limited to, ATP-competitive CaMKII inhibitors. In some embodiment, the health condition includes a chronic or perpetuating health condition. In some embodiments, the chronic or perpetuating health condition includes, but is not limited to, conditions affecting brain function having an impact on brain activities in a subject. It is known that mechanisms of LTP impairment can be alleviated by inhibition of enzymatic CaMKII activity. However, many CaMKII inhibitors are also known to directly inhibit LTP, causing limitations to any therapeutic use of such CaMKII inhibitors that can penetrate the brain. As disclosed herein, some CaMKII inhibitors are non-ATP-competitive and directly inhibit LTP induction, but other CaMKII inhibitors that are ATP-competitive do not. This observations allows therapeutic use of ATP-competitive CaMKII inhibitors that can penetrate the brain while preserving LTP induction.

In some embodiments and further to paragraph above, compositions containing CaMKII inhibitors of use herein include CaMKII inhibitors that do not inhibit or prevent induction of long-term potentiation (LTP) at the hippocampal CA1 synapse and can include any ATP-competitive CaMKII inhibitor. In accordance with these embodiments, ATP-competitive CaMKII inhibitors can include, but are not limited to, AS10010A5, AS100283, AS100397 (referred to herein as AS105, AS283, or AS397 respectively (Allosteros Therapeutics, Inc.)), bosutinib, sunitinib, RA306, RA608, Scios-15b, dainippon, dainipponB:25, 3′,4′-dihydroflavonol (DiOHF), rimacalib/smp-114, ruxolitinib, barictinib, other Janus Kinase (JAK) inhibitors, and the like, and any combination thereof. It is understood by the instant disclosure that other known CaMKII inhibitor classes can block LTP induction; and are not of use for embodiments disclosed herein, at least if the agent is capable of penetrating the brain. It is contemplated herein that CaMKII inhibitors of use to treat, prevent, eliminate, or ameliorate a health condition disclosed herein are ATP competitive CaMKII inhibitors and not an ATP non-competitive CaMKII inhibitors. In certain embodiments, use of a composition including, but not limited to, an ATP-competitive CaMKII inhibitor contemplated herein can circumvent or overcome a need for activities related to autophosphorylation of threonine 286 (Thr 286) or equivalent thereof, of the CaMKII.

In certain embodiments and further to paragraphs [0006]-[0007] above, compositions disclosed herein can include one or more ATP-competitive CaMKII inhibitor for preserving CaMKII structural functions including, but not limited to CaMKII binding to N-methyl-D-aspartate (NMDA)-type glutamate receptor (NMDAR) subunit GluN2B. The NMDA receptor is a receptor of glutamate, a primary excitatory neurotransmitter in the human brain. It plays an integral role in synaptic plasticity, a neuronal mechanism believed to be the basis of memory formation. In some embodiments, compositions disclosed herein can include one or more ATP-competitive CaMKII inhibitor for inducing or restoring long-term potentiation (LTP) in the brain of a subject. LTP and long-term depression (LTD) are involved with learning, memory, and cognition. In accordance with these embodiments, these functions can be impaired or affected in subjects having brain-related or brain-affected conditions such as Alzheimer's Disease (AD), Downs Syndrome (DS), stroke (focal cerebral ischemia), traumatic brain injury (TBI), global cerebral ischemia, other condition that impairs cognition, learning and/or memory, or other health condition related to CaMKII structural functions disclosed herein. In some embodiments, the health condition includes a chronic or progressive health condition or syndrome; for example, a chronic or progressive health condition that affects brain function; for example, impaired learning, impaired memory, and/or impaired cognition.

In some embodiments and further to paragraphs [0006]-[0008] above, one or more ATP-competitive CaMKII inhibitor contemplated herein can be used alone or in combination with other agents to treat a subject. In certain embodiments, another agent of use in combination with an ATP-competitive CaMKII inhibitor can include, but is not limited to, a pharmaceutically acceptable composition containing one or more agents of use to treat β-amyloid and/or reduce or prevent β-amyloid plaque formation or treat adverse activities of Aβ precursor protein (APP), oligomeric amyloid-β peptide (Aβ), or combination thereof. In some embodiments, a combination pharmaceutical composition including, but not limited to, at least one ATP-competitive CaMKII inhibitor and at least one anti-β-amyloid agent is contemplated of use to prevent, ameliorate, treat, or eliminate a health condition in a subject. In certain embodiments, these combination compositions can be used for short or prolonged treatment and avoid adverse effects on LTP.

In certain embodiments and further to paragraphs [0006]-[0009] above, a subject contemplated herein is a human subject or other mammalian subject. In accordance with these embodiments, a human subject can include a fetus, an infant, a toddler, a child, an adolescent, a young adult, an adult, or an elderly adult. In other embodiments, the human subject can include an adolescent, young adult, an adult or elderly adult.

In some embodiments and further to paragraphs [0006]-[0010] above, methods for administering compositions disclosed herein to a subject can include any suitable mode of administration. In some embodiments, methods of administering compositions can include intravenous, intranasal, oral, subcutaneous, intramuscular, by inhalation, timed-release or other suitable mode of administration.

In certain embodiments and further to paragraphs [0006]-[0011] above, the present disclosure provides kits for storing or transporting compositions or combination compositions disclosed herein and for using to treat a subject contemplated herein and for practicing any of the methods disclosed herein. In some embodiments, kits disclosed herein can include combination compositions for administration to a subject in need thereof, and at least one container, and/or instructions.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIG. 1A represents a schematic diagram of photoactivable CaMKII (paCaMKII) in its basal and photoactivated states, according to various embodiments of the instant disclosure.

FIG. 1B represents exemplary confocal microscopy images of a cell line co-expressing paCaAMKII (wild type (WT) or mutants) and GluN2B (2BC) before and after photoactivation, according to various embodiments of the instant disclosure.

FIG. 1C represents a bar graph depicting Pearson's correlation (correlation index) of wild type (WT) or mutant paCaMKII and GluN2B (mCh-2BC) after photoactivation, according to various embodiments of the instant disclosure.

FIG. 1D represents exemplary confocal microscopy images of dissociated rat hippocampal cultures expressing WT or mutant paCaMKII before and after photoactivation, according to various embodiments of the instant disclosure.

FIG. 1E represents a bar graph depicting fold change of synaptic enrichment in dissociated hippocampal cultures expressing WT or mutant paCaMKII after photoactivation, according to various aspects of the disclosure.

FIG. 1F represents a bar graph depicting fold change of synaptic enrichment after paCaMKII photoactivation in WT and GluN2B^{ΔCaMKII} mice according to various embodiments of the instant disclosure.

FIG. 1G represents two plots depicting changes in dendritic spine regions after photoactivation in hippocampal cultures overexpressing WT or mutant paCaMKII (left plot) and in hippocampal cultures obtained from WT and GluN2B^{ΔCaMKII} mice (right plot), according to various aspects of the disclosure.

FIG. 2A represents a schematic of CaMKII in its basal and active state with binding sites for GluN2B and an inhibitor as indicated, according to various embodiments of the instant disclosure.

FIG. 2B represents an illustrative immunoblot (top image) and quantification represented by a graph (bottom image) of in vitro kinase reactions measuring CaMKIIα phosphorylation of GluA1 S831 in the presence of increasing concentrations of a CaMKIIα inhibitor (AS283), according to various embodiments of the instant disclosure.

FIG. 2C represents an illustrative immunoblot and quantification in a bar graph of bound CaMKII to GluN2B as induced by Ca²⁺/CaM and ADP with or without the inhibitors AS283 and tatCN21 (a control CaMKII inhibitor that inhibits LTP, by interfering with CaMKII interaction with the GluN2B subunit of the N-methyl-D-aspartate (NMDA)-type glutamate receptor (NMDAR)), according to various embodiments of the instant disclosure.

FIG. 2D represents illustrative confocal microscopy images and quantification in a graph of CaMKII and GluN2B co-localization in a transduced cell line before (Pre) and after (Stim) treatment with ionomycin and in the presence or absence of an inhibitor, AS283, according to various embodiments of the instant disclosure.

FIG. 2E represents illustrative confocal microscopy images of dissociated rat hippocampal cultures expressing GFP-CaMKII and mCh-PSD95 intrabody in the presence of an inhibitor (AS283) before and after chemical LTP (top images) and quantification in a graph of synaptic enrichment (bottom image), according to various embodiments of the instant disclosure.

FIG. 3A represents an illustrative plot of LTP induction in hippocampal slices in WT mice in the presence or absence of CaMKII inhibitors, AS283 or tatCN21 and a representative plot thereof, according to various embodiments of the instant disclosure.

FIG. 3B represents a bar graph quantifying LTP induction in WT mice in the presence or absence of CaMKII inhibitors, AS283 and tatCN21, according to various embodiments of the instant disclosure.

FIG. 3C represents an illustrative graph of LTP induction in hippocampal slices from mutant mice expressing T286A CaMKII in the presence of absence of an CaMKII inhibitor, AS283 and a representative graph correlating to these conditions, according to various embodiments of the instant disclosure.

FIG. 3D represents a plot quantifying LTP induction in hippocampal slices from mutant mice expressing T286A CaMKII in the presence of absence of the CaMKII inhibitor, AS283, according to various embodiments of the instant disclosure.

FIG. 3E represents an illustrative immunoblot (top image) and quantification illustrated by bar graph (bottom image) of bound CaMKIIα to immobilized WT GluN2B after activation of CaMKIIα with Ca²⁺/CaCM in the presence or absence of ATP or AS283, according to various embodiments of the instant disclosure.

FIG. 3F represents an illustrative immunoblot (top image) and quantification by bar graph (bottom image) of bound CaMKIIα to immobilized mutant GluN2B (S1303A) after activation of CaMKIIα with Ca²⁺/CaCM in the presence or absence of ATP or AS283, according to various embodiments of the instant disclosure.

FIG. 4A represents an illustrative schematic of a pharmacogenetic method of ATP-competitive CaMKII inhibition demonstrating that enlargement or accommodation of the CaMKII ATP-binding pocket by the F89G mutation allows for selective binding of a mutant-specific ATP-competitive inhibitor NM-PP1, according to various embodiments of the instant disclosure.

FIG. 4B represents an illustrative immunoblot of in vitro kinase reactions measuring activity of WT and mutant CaMKII (K42M and F89G mutants) (measured by phosphorylation of GluA1 S831) in the presence or absence of a mutant CaMKII specific (F89G) inhibitor, NM-PP1, according to various embodiments of the instant disclosure.

FIG. 4C represents illustrative confocal microscopy images (top images) and bar graph plots (bottom image) of cells co-expressing GFP-CaMKII F89G and mCherry-2BC in the presence of vehicle or 10 μM NM-PP1 before and after ionomycin stimulation, according to various embodiments of the instant disclosure.

FIG. 4D represents confocal microscopy images (top image) and bar graph image plots (bottom image) of dissociated rat hippocampal cultures expressing GFP-CaMKII (WT, K42M, or F89G) and mCh-PSD95 intrabody before and after cLTP, according to various embodiments of the instant disclosure.

FIG. 4E represents confocal microscopy images (top image) and bar graph plots (bottom image) of dissociated rat hippocampal cultures expressing GFP-CaMKII (WT, K42M, or F89G) and mCh-PSD95 intrabody in the presence of 10 μM NM-PP1 before and after cLTP, according to various embodiments of the instant disclosure.

FIG. 5A represents a schematic image of a molecular replacement approach (top and middle images) and a representative image of an acute hippocampal slice expressing AAV-mScarlet-CaMKII F89G (red) and labelled with DAPI (blue) (bottom image), according to various embodiments of the instant disclosure.

FIG. 5B represents an illustrative plot of LTP induction in hippocampal slices from CaMKIIα knockout mice injected with AAV-mScarlet (mock) in the presence or absence of NM-PP1 and representative graphs thereof, according to various embodiments of the instant disclosure.

FIG. 5C represents a plot quantifying synaptic response after LTP induction in hippocampal slices from CaMKIIα knockout mice injected with AAV-mScarlet (mock) in the presence or absence of NM-PP1, according to various embodiments of the instant disclosure.

FIG. 5D represents an illustrative plot of LTP induction in hippocampal slices from CaMKIIα KO mice injected with AAV-mScarlet-CaMKII-F89G in the presence or absence of NM-PP1 and representative graphs thereof, according to various embodiments of the instant disclosure.

FIG. 5E represents a bar graph plot quantifying synaptic response after LTP induction in hippocampal slices from CaMKIIα knockout mice injected with AAV-mScarlet-CaMKII-F89G in the presence or absence of NM-PP1, according to various embodiments of the instant disclosure.

FIG. 6A represents exemplary photographic images of GFP-paCaMKII T286A and mCherry cell fill expressed in cultured hippocampal neurons before and after blue light stimulation and in the presence (right panels) or absence (left panels) of the CaMKII inhibitor AS283, according to various embodiments of the instant disclosure.

FIG. 6B represents a bar graph illustrating fold change of paCaMKII T286A synaptic enrichment values in the presence or absence of AS283 compared to paCaMKII WT (from FIG. 1E), according to various embodiments of the instant disclosure.

FIG. 6C represents a bar graph illustrating changes in dendritic spine regions measured after photoactivation of paCaMKII T286A in the presence or absence of AS283 compared to paCaMKII WT (from FIG. 1F), according to various embodiments of the instant disclosure.

FIG. 6D represents illustrative images of GFP-paCaMKII F89G and mCherry cell fill expressed in cultured hippocampal neurons before and after blue light stimulation in the presence or absence of NM-PP1, according to various embodiments of the instant disclosure.

FIGS. GE-6F represents a bar graph illustrating changes in synaptic enrichment (6E) and dendritic spine regions (6F) measured after photoactivation of paCaMKII F89G in the presence or absence of NM-PP1 compared to paCaMKII WT (from FIG. 1E and FIG. 1F, respectively), according to various embodiments of the instant disclosure.

FIG. 7A represents illustrative photographic images (top image) and graph over a preselected period (bottom image) of GFP-paCaMKII WT and mCherry-2BC expressed before and after blue light stimulation, according to various embodiments of the instant disclosure.

FIG. 7B represents a schematic of blue light-induced paCaMKII pulldown to immobilized GST-2BC, according to various embodiments of the instant disclosure.

FIG. 7C represents an illustrative immunoblot (top image) and a graphic representation of quantification (bottom image) illustrating levels of CaMKII-F89G pulled down with GST-2BC after blue light stimulation in the presence or absence of ADP, according to various embodiments of the instant disclosure.

FIG. 7D represents an illustrative schematic of K42M and I205K CaMKII mutations affect on enzymatic activity and the induction of GluN2B binding, according to various embodiments of the instant disclosure.

FIG. 7E represents illustrative photographic images of dissociated mouse WT and GluN2B^{ΔCaMKII} hippocampal cultures expressing GFP-paCaMKII WT and mCherry cell fill before and after photoactivation, according to various embodiments of the instant disclosure.

FIG. 8A represent images of the chemical structures of AS105 and AS283, according to various embodiments of the instant disclosure.

FIG. 8B represent immunoblots of in vitro kinase reactions at 30° C. with increasing ATP concentration and measuring purified CaMKIIα phosphorylation of GST-GluA1 S831, according to various embodiments of the instant disclosure.

FIG. 8C represents a graph illustrating quantifying purified CaMKIIα phosphorylation of GST-GluA1 S831 in the presence of increasing ATP concentrations, according to various embodiments of the instant disclosure.

FIG. 8D represents an immunoblot (top image) and bar graph (bottom image) of bound purified CaMKIIα to GST-GluN2B-c induced by Ca²⁺/CaM without nucleotide (None), in the presence of ADP, or in the presence of AS105, according to various embodiments of the instant disclosure.

FIG. 8E represents exemplary photographic representations of confocal microscopy images and correlation indices of cells (HEK293) cells co-expressing CaMKII and mCherry-2BC before and after ionomycin treatment (Stim) in the presence or absence of AS105, according to various embodiments of the instant disclosure.

FIG. 8F represents exemplary photographic representations of confocal microscopy images (top images) and correlation indices represented in graphs (bottom images) of dissociated rat hippocampal cultures expressing GFP-CaMKII and mCh-PSD95 intrabody in the presence of AS105 before and after cLTP, according to various embodiments of the instant disclosure.

FIG. 9A represents an illustrative graph of LTP induction in hippocampal slices from WT mice in the presence or absence of a CaMKII inhibitor, AS283 and an exemplary recording therefore thereof, according to various embodiments of the instant disclosure.

FIG. 9B represents a bar graph quantifying LTP induction in hippocampal slices from WT the presence or absence of a CaMKII inhibitor, AS283, according to various embodiments of the instant disclosure.

FIG. 9C represents an illustrative immunoblot (top images) and a bar graph (bottom image) of quantification of CaMKII T286 phosphorylation in dissociated hippocampal cultures after induction of chemical LTP (cLTP) and in the presence of inhibitor, AS283, according to various embodiments of the instant disclosure.

FIG. 9D represents an illustrative immunoblot (top image) and bar graph (bottom image) quantification of in vitro kinase reactions with purified CaMKII and GST-2BC measuring S1303 phosphorylation and inhibition by AS283, according to various embodiments of the instant disclosure.

FIG. 10A represents an illustrative immunoblot (top image), a bar graph (middle image) and a table representing percent activity (bottom image) quantification of in vitro kinase reactions in ATP measuring CaMKIIα WT and F89G phosphorylation of T286, according to various embodiments of the instant disclosure.

FIG. 10B represents photographic images regarding confocal microscopy images of HEK293 cells co-expressing GFP-CaMKII WT (top images) or GFP-CaMKII K42M (bottom images) and mCherry-2BC in the presence of control (vehicle) or NM-PP1 before and after ionomycin stimulation, according to various embodiments of the instant disclosure.

FIG. 10C represents illustrative immunoblots (top image) and bar graph (bottom image) quantification of CaMKII-F89G binding to GDT-GluN2B-c induced by Ca2+/CaM in the presence of ADP or NM-PP1, according to various embodiments of the instant disclosure.

FIG. 11A represents an illustrative schematic of a molecular replacement approach (top images) and representative image of acute hippocampal slice expressing AAV-mScarlet (Mock) (red) and labelled with DAPI (blue) (bottom image), according to various embodiments of the instant disclosure.

FIG. 11B represents an illustrative bar graph depicting data illustrated in FIG. 5C, according to various embodiments of the instant disclosure.

FIG. 11C represents an illustrative bar graph depicting data illustrated in FIG. 5E, according to various embodiments of the instant disclosure.

FIG. 11D represents an illustrative plot of LTP induction in hippocampal slices from CaMKIIα KO mice injected with AAV-mScarlet-CaMKIIF89G (e.g., mutated CaMKII with expanded ATP binding pocket that is selectively antagonized by NM-PP1 (WT CaMKII is not affected by NM-PP1) in the presence or absence of NM-PP1, according to various embodiments of the instant disclosure.

FIG. 11E is a representative bar graph quantifying LTP induction in hippocampal slices from CaMKIIα KO mice injected with AAV-mScarlet-CaMKIIF89G in the presence or absence of NM-PP1, according to various embodiments of the instant disclosure.

FIG. 12 represents an illustrative schematic of CaMKII enzymatic and structural functions and their roles in LTP induction, according to various aspects of the disclosure.

FIG. 13 represents an illustrative scatter plot quantifying CaMKII movement to excitatory synapses in hippocampal neurons in dissociated culture in the presence or absence of a soluble oligomeric amyloid β-peptide alone or with various ATP-competitive CaMKII inhibitors as indicated, according to various embodiments of the instant disclosure.

FIG. 14A represents a bar graph of LTP induction in hippocampal slices from CaMKII T285A mutant mice in the presence or absence of two different ATP-competitive CaMKII inhibitors (AS397 or Ruxo), according to various embodiments of the instant disclosure.

FIG. 14B represents a bar graph quantifying LTP induction in hippocampal slices from CaMKII T285A mutant mice in the presence or absence of two different ATP-competitive CaMKII inhibitors (e.g., AS397 or Ruxo), according to various embodiments of the instant disclosure.

FIG. 15 represents exemplary photographic images of confocal microscopy images of dissociated hippocampal neurons illustrating light induced movement of paCaMKII to excitatory synapses (top images), quantification of synaptic enrichment (bottom left image) and quantification of spine growth (bottom right image) in the presence and absence of an APT-competitive inhibitor (AS397), according to various embodiments of the instant disclosure.

FIG. 16A represents a schematic of an in vitro binding reaction between GST-GluN2Bc and purified CaMKIIα under certain experimental conditions according to various embodiments of the instant disclosure.

FIG. 16B represents illustrative immunoblots (bottom image) and a bar graph (top image) of quantification of an in vitro binding reaction with GST-GluN2Bc and purified CaMKIIα in the presence of different ATP competitive CaMKII inhibitors (e.g., AS397, AS283 or Ruxolitinib), according to various embodiments of the instant disclosure.

FIG. 16C represents illustrative fluorescent images (top images) and graphical representations (bottom images) demonstrating co-localization of GFP-CaMKII (green) with a membrane targeted mCherry-GluN2B C-tail (red) in HEK 293 cells before or after treatment with ionomycin and in the presence or absence of CaMKII inhibitors, JAK inhibitors (AS397 or Ruxolitinib), according to various embodiments of the instant disclosure.

FIG. 16D represents an illustrative bar graph demonstrating quantifying net change in CaMKII/GluN2B co-localization after drug incubation and ionomycin treatment of cells found experiments of representative images in FIG. 16C, according to various embodiments of the instant disclosure.

FIG. 16E represents a graph of a time course of CaMKII co-localization with GluN2B in cells found in experiments of representative images in FIG. 16C, according to various embodiments of the instant disclosure.

FIG. 17 represents an illustrative graph (top image) and a bar graph of quantification (bottom image) of LTP induction and maintenance in WT hippocampal slices in the presence or absence of AS397, according to various embodiments of the instant disclosure.

### DEFINITIONS

Terms, unless defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis, or therapy is desired, for example, humans, or other mammals.

### DETAILED DESCRIPTION OF THE INVENTION

In the following sections, certain exemplary compositions and methods are described to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Long term potentiation (LTP) is impaired by several processes and events; for example, (1) pharmacological CaMKII inhibition with two different classes of mechanistically distinct inhibitors, (2) by knockout of the CaMKIIα isoform that is predominant in brain, (3) by introducing a mutation into CAMKII that prevents ATP binding, or (4) by a T286A mutation in CAMKII that prevents the T286 autophosphorylation (pT286) that generates Ca²⁺-independent “autonomous” CaMKII kinase activity. However, all these LTP-inhibiting interventions with enzymatic CaMKII activity also interfere with CaMKII binding to the N-methyl-D-aspartate (NMDA) receptor (NMDAR) subunit GluN2B (subunit involved in synapse development) which is problematic when seeking a specific inhibitor for this molecule. For example, (i) calmodulin-competitive inhibitors such as KN93 or KN62 prevent the stimulus that induces both activity and GluN2B binding, (ii) the peptide inhibitors tatCN21, tatCN190, AIP, and AC3-I bind to the CaMKII T-site, which is also the binding site for GluN2B, (iii) the K42M and K42R mutants prevent nucleotide binding to CaMKII, which is a requirement not only for activity but also for effective GluN2B binding, and (iv) while the T286A mutation does not completely block CaMKII binding to GluN2B, it significantly impairs the stimulation-induced binding of CaMKII to GluN2B within cells and the resulting synaptic CaMKII accumulation in neurons.

In certain embodiments and further to paragraph above, mechanisms of LTP impairment can be alleviated by inhibition of enzymatic CaMKII activity. Although LTP impairment can be alleviated by inhibition of enzymatic CaMKII activity, some CaMKII inhibitors are known to inhibit LTP, causing limitations to any therapeutic use of these CaMKII inhibitors. In certain cases, short- and long-term use of these CaMKII inhibitors that affect LTP is discouraged or impermissible. Certain CaMKII inhibitors contemplated of use herein can relieve LTP impairment while inhibiting CaMKII and penetrate the blood-brain barrier. In certain embodiments, these agents that do not interfere with LTP can be used to treat the subject and in particular, can be used in treating the brain and brain functions of the subject. Certain CaMKII inhibitors that are non-ATP-competitive directly inhibit LTP induction, but as disclosed herein CaMKII inhibitors that are ATP-competitive do not. Therefore, in certain embodiments, therapeutic use of ATP-competitive CaMKII inhibitors able to penetrate the brain is contemplated of use herein to treat, ameliorate, and/or prevent LTP impairment and/or restore LTP function while inhibiting or eliminating adverse CaMKII activities.

Embodiments of the instant disclosure and further to paragraphs [0085]-[0086] above, relate to new therapeutic uses for certain Ca²⁺/Calmodulin-Dependent Protein Kinase II (CaMKII) inhibitors for preventing, ameliorating, reversing, treating, and/or curing a health condition. In certain embodiments, the health condition includes, but is not limited to, any health condition concerning CaMKII. In some embodiments, the health condition concerns a condition of the brain and targeting the brain with one or more ATP-competitive CaMKII inhibitor to prevent, ameliorate or treat the health condition. In accordance with these embodiments, the health condition can include preservation and/or repair of LTP to improve learning, memory and/or cognition in a subject in need thereof. In certain embodiments, compositions and methods disclosed herein can be used to restore LTP mechanisms or functions for example, impaired in a subject by Aβ (e.g., AD related, DS or the like) or other similar mechanism or agent.

In some embodiments and further to paragraphs [0086]-[0087] above, CaMKII inhibitors contemplated of use herein inhibit certain CaMKII activities while preserving structural CaMKII functions. In certain embodiments, CaMKII inhibitors of use herein inhibit CaMKII enzymatic activity. In some embodiments, CaMKII inhibitors of use herein circumvent CaMKII enzymatic activity (e.g., kinase activity) related to autoregulation of its structural role through a T286 autophosphorylation; for example, by directly initiating CaMKII structural functions in a manner outside of its autophosphorylation T286 role. In some embodiments, CaMKII autophosphorylation at T286 can be needed to induce structural functions, but this phosphorylation can be circumvented by providing ATP-competitive CaMKII inhibitors that enhance CaMKII/GluN2B binding, for example.

Some embodiments and further to paragraphs [0085]-[0088] above, methods for method for preventing, treating, ameliorating, or eliminating a health condition in a subject can include administering to the subject a composition having one or more ATP-competitive CaMKII inhibitors, where the composition preserves CaMKII structural function while preventing, treating, ameliorating, or eliminating a health condition in a subject. In other embodiments, the health condition can include a health condition affecting the brain of the subject. In other embodiments, the health condition includes a health condition affecting one or more of learning, memory, and cognition in the subject. In some embodiments, the brain condition is a side effect of a health condition, and the health condition includes one or more of Down Syndrome (DS), Alzheimer's Disease (AD), a cerebral ischemia, a traumatic brain injury (TBI), other brain condition or brain injury, or a combination thereof. In other embodiments, the health condition includes a neurological condition due to adverse activities of Aβ precursor protein (APP), oligomeric amyloid-β peptide (Aβ), or combination thereof. In yet other embodiments, the one or more ATP-competitive CaMKII inhibitors include a JAK inhibitor or a small molecule. In some embodiments, methods can further include administering a standard treatment for improving learning, memory, and cognition in the subject at least one of: in combination with, before, during, or after administering the one or more ATP-competitive CaMKII inhibitors to the subject. In some embodiments, the one or more ATP-competitive CaMKII inhibitors or JAK inhibitors do not reduce or preserve long-term potentiation (LTP) mechanisms in the subject; optionally, where the one or more ATP-competitive CaMKII inhibitors preserve CaMKII movement to synapses and preserves CaMKII synaptic activity.

In some embodiments and further to paragraphs [0085]-[0089] above, the one or more ATP-competitive CaMKII inhibitors restore LTP mechanisms impaired by β-amyloid peptide (Aβ) in a subject compared to a subject having impaired LTP mechanisms due to β-amyloid peptide (Aβ) not treated with the one or more ATP-competitive CaMKII inhibitors. In yet other embodiments, the one or more ATP-competitive CaMKII inhibitors do not inhibit CaMKII binding to N-methyl-D-aspartate (NMDA)-type glutamate receptor (NMDAR) subunit GluN2B.

In some embodiments and further to paragraphs [0085]-[0090] above, methods for preventing, treating, ameliorating, repairing/restoring or eliminating a health condition in a subject can include administering to the subject a composition of one or more ATP-competitive CaMKII inhibitors, alone or in combination with at least one additional agent wherein the one or more CaMKII inhibitors cross the blood-brain barrier, do not inhibit, or prevent induction of long-term potentiation (LTP) and do not prevent CaMKII inhibition in the brain of the subject while preventing, treating, ameliorating, or eliminating the health condition in the subject. In certain embodiments, the health condition includes a condition or trauma that interferes with, or reduces learning, memory and/or cognitive function in the subject. In some embodiments, the composition or a combination composition does not reduce learning, memory, or cognitive functions in the subject. In certain embodiments, administering the composition or combination composition can include administering the composition or combination composition one time, for a predetermined period, continuously, for long-term treatment of the subject, or for the remainder of the life of the subject. In some embodiments, administering the composition or combination composition can include administering the composition or combination composition for long term or for a continuous treatment period three times daily, two times daily, daily, every other day, twice weekly, weekly, bi-monthly, or monthly.

In some embodiments and further to paragraphs [0085]-[0091] above, ATP-competitive CaMKII inhibitors of use herein can include, but are not limited to, any known ATP-competitive CaMKII inhibitor in the art. In accordance with these embodiments, ATP-competitive CaMKII inhibitors can include, but are not limited to, AS100105, AS100283, AS100397 (also referred to as AS105, AS283, or AS397 respectively (Allosteros Therapeutics, Inc.)), bosutinib, sunitinib, RA306, RA608, GS-680, Scios-15b, diaippon, dainipponB:25, 3′,4′-dihydroflavonol (DiOHF), NP202), rimacalib/smp-114, ruxolitinib, barictinib, other Janus Kinase (JAK) inhibitors, and the like. In certain embodiments, ATP-competitive CaMKII inhibitors can include, but are not limited to, AS100283 (also referred to as AS283), AS105, or a combination thereof. In certain embodiments, the one or more ATP-competitive CaMKII inhibitors include AS100283, AS100397, ruxolitinib, barictinib or a combination thereof. In other embodiments, ATP-competitive CaMKII inhibitors can include, but are not limited to, an antibody or monoclonal antibody that preserve LTP but bind to CaMKII. In yet other embodiments, ATP-competitive CaMKII inhibitors can include, but are not limited to, ruxolitinib (Rux), barictinib, or a combination thereof. It is contemplated herein that CaMKII inhibitors of use to treat, prevent, eliminate, or ameliorate a health condition disclosed is an ATP competitive CaMKII inhibitor and not an ATP non-competitive CaMKII inhibitor. In certain embodiments, an ATP-competitive CaMKII inhibitor of use herein can circumvent or eliminate the need for activities related to autophosphorylation of threonine 286 (Thr 286) or equivalent thereof, of the CaMKII.

In some embodiments and further to paragraphs [0085]-[0092] above, at least one additional agent comprises an anti-β-amyloid peptide agent or other anti-amyloid agent treatment; optionally wherein the agent comprises omega-3 fatty acids, guanidine salts, dimethyl sulfoxide (DMSO), vitamin C, lecanemab, donanemab, or a combination thereof. In other embodiments, a combination agent with compositions disclosed herein can include serine, for example, D-serine. In certain embodiments, a composition for preserving or restoring LTP function in a subject, can include one or more ATP-competitive CaMKII inhibitors, and at least one anti-β-amyloid peptide agent or other anti-amyloid agent. In other embodiments, the one or more ATP-competitive CaMKII inhibitors comprises one or more of AS100105, AS100283, AS100397, bosutinib, sunitinib, RA306, RA608, Scios-15b, diaippon, dainipponB:25, 3′,4′-dihydroflavonol (DiOHF), rimacalib/smp-114, ruxolitinib, barictinib, other Janus Kinase (JAK) inhibitors, other ATP CaMKII inhibitors or a combination thereof. In yet other embodiments, a composition or combination composition can further include at least one pharmaceutically acceptable excipient. In other embodiments, kits contemplated herein can include a combination composition and at least one container.

In certain embodiments and further to paragraphs [0085]-[0093] above, ruxolitinib and/or barictinib which are known generally as Janus Kinase (JAK) inhibitors commonly used to treat certain cancers, hair loss and other conditions were surprisingly found to have ATP competitive CaMKII inhibitor activity while preserving LTP induction. In addition, it was discovered herein that these JAK inhibitors cross the blood-brain barrier and can act as ATP competitive CaMKII inhibitors and inhibit adverse CaMKII activities in the brain of a subject and preserve, treat, prevent, restore, or improve learning, memory, and cognition or other brain activities in a subject. In some embodiments, compositions including at least one of ruxolitinib and/or barictinib and optionally, other agents, can be used to treat, prevent, restore function, or ameliorate a brain-related, brain growth, brain function, and/or other health condition in a subject.

In certain embodiments and further to paragraphs [0085]-[0094] above, administering a composition disclosed herein to a subject can be a single dose administration, administering a composition disclosed herein for a predetermined short (e.g., less than a week, less than months or less than three months) or administering a composition disclosed herein for a prolonged (e.g., three months or more, six months or more, one year or more or continuous) period. In certain embodiments, a subject can be treated for less than or for the entire time the subject is experiencing the health condition or effects of the health condition such as adverse effects on learning, memory and/or cognition.

In certain embodiments and further to paragraphs [0085]-[0095] above, compositions and methods disclosed herein can be used to treat a subject contemplated herein in combination with standard treatments. In some embodiments, compositions and methods disclosed herein can be used to treat a subject contemplated herein in combination with an anti-plaque, an anti-amyloid (e.g., anti-amyloid β), an agent against adverse activities of Aβ precursor protein (APP), an agent against adverse activities of oligomeric amyloid-β peptide (Aβ), or combination thereof treatment for a health condition contemplated herein (e.g., AD, DS, diabetes, etc.).

In certain embodiments and further to paragraphs [0085]-[0096] above, compositions disclosed herein can include one or more ATP-competitive CaMKII inhibitor for preserving CaMKII structural functions including, but not limited to, CaMKII binding to NMDA-type glutamate receptor (NMDAR) subunit GluN2B. In some embodiments, compositions disclosed herein can include one or more ATP-competitive CaMKII inhibitor for inducing or restoring long-term potentiation (LTP) in the brain of a subject. LTP and long-term depression (LTD) are involved with learning, memory, and cognition. In accordance with these embodiments, these functions are impaired or affected in subjects having brain-related conditions such as Alzheimer's Disease (AD), Downs Syndrome (DS), stroke (focal cerebral ischemia), traumatic brain injury, global cerebral ischemia, other condition that impairs cognition, learning and/or memory, or other health condition related to CaMKII structural functions disclosed herein. In some embodiments, the health condition includes a chronic or progressive health condition or syndrome; for example, a chronic or progressive health condition that affects brain function and/or brain growth; for example, leading to impaired learning, impaired memory, and/or impaired cognition. In some embodiments, compositions disclosed herein can be administered to a subject in need thereof and tasks performed or behavioral observations made and administration of the composition can be adjusted as needed based on observations of tasks performed or behavioral indicators.

In some embodiments and further to paragraphs [0089]-[0097] above, one or more ATP-competitive CaMKII inhibitor contemplated herein can be used alone or in combination with other agents to treat a subject. In certain embodiments, another agent of use in combination with an ATP-competitive CaMKII inhibitor can include, but is not limited to, a pharmaceutically acceptable composition containing one or more agents of use to treat production of β-amyloid and/or reduce or prevent β-amyloid plaque formation or adverse effects thereof. In some embodiments, a combination pharmaceutical composition including, but not limited to, at least one ATP-competitive CaMKII inhibitor and at least one anti-β-amyloid agent is contemplated of use to prevent, ameliorate, treat, restore, or eliminate a health condition in a subject. In certain embodiments, these combination compositions can be used for short or prolonged treatment and avoid adverse effects on LTP, preserve LTP or reverse adverse effects on LTP in a subject.

In certain embodiments and further to paragraphs [0085]-[0098] above, a subject contemplated herein is a human subject or other mammalian subject. In accordance with these embodiments, a human subject can include a fetus, an infant, a toddler, a child, an adolescent, a young adult, an adult, or an elderly adult. In other embodiments, the human subject can include a child, an adolescent, young adult, an adult or elderly adult.

In some embodiments and further to paragraphs [0085]-[0099] above, methods for administering compositions disclosed herein to a subject can include any suitable mode of administration. In some embodiments, methods of administering compositions can include intravenous, intranasal, by inhalation, oral, subcutaneous, slow-release formulations, or other suitable mode of administration. In some embodiments, methods of administering compositions can include two or more different modes of administration for a single agent or combination of agents.

In certain embodiments and further to paragraphs [0085]-[0100] above, the present disclosure provides kits for storing compositions disclosed herein and for use in practicing any of the methods disclosed herein. In some embodiments, kits disclosed herein can include combination compositions for administration to a subject in need thereof.

In certain embodiments and further to paragraphs [0085]-[0101] above, pharmaceutical compositions are contemplated. In accordance with these embodiments, pharmaceutical compositions can include one or more ATP-competitive CaMKII inhibitor disclosed herein. In some embodiments, pharmaceutical compositions herein can include one or more ATP-competitive CaMKII inhibitor disclosed herein and at least one pharmaceutically acceptable excipient or carrier. As used herein, the term “pharmaceutically acceptable carrier” can refer to solvents, dispersion media, coatings, antibacterial agents, antifungal agents, isotonic and absorption delaying agents, or the like that are physiologically compatible. Pharmaceutically acceptable carriers suitable for use herein, include, but are not limited to, buffers that are well known in the art, and can be phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

In some embodiments and further to paragraphs [0085]-[0102] above, pharmaceutical compositions for use herein can be formulated for parenteral administration, such as intravenous or intravascular, bolus infusion, intrarenal introduction, intracerebroventricular injection, intra-cisterna magna injection, intra-parenchymal injection, or a combination thereof. In some embodiments, pharmaceutical compositions for use herein can be formulated for infusion of one or more ATP-competitive CaMKII inhibitor. In some embodiments, pharmaceutical compositions for use herein be formulated for parenteral/infusion administration can include pharmaceutically acceptable carriers including sterile liquids, such as water and oil, including those of petroleum, animal, vegetable, or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. In some embodiments, pharmaceutical compositions for use herein can further include additional ingredients, for example preservatives, buffers, tonicity agents, antioxidants and stabilizers, nonionic wetting or clarifying agents, viscosity-increasing agents, and the like. In some embodiments, pharmaceutical compositions described herein can be packaged in single unit dosages or in multi-dosage forms.

In some embodiments and further to paragraphs [0085]-[0103] above, formulations suitable for parenteral/infusion administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents. In accordance with some embodiments herein, aqueous solutions can be suitably buffered (e.g., a pH of from 3 to 9). The preparation of suitable parenteral/infusion formulations for use herein under sterile conditions can be readily accomplished by standard pharmaceutical techniques well known to those skilled in the art.

In some embodiments and further to paragraphs [0085]-[0104] above, pharmaceutical compositions herein can further include one or more pharmaceutically acceptable salts. Non-limiting examples of pharmaceutically acceptable salts include acid addition salts (formed from a free amino group of a polypeptide with an inorganic acid, or an organic acid. In some embodiments, the salt formed with the free carboxyl groups is derived from an inorganic base, or an organic base. In some embodiments, any of the pharmaceutical compositions herein can be used in therapeutic applications, for example, treating a disease and/or a disorder in human patients, which are disclosed herein. In some embodiments, any of the pharmaceutical compositions disclosed herein can be used in therapeutic applications, for example, treating a brain disorder.

In certain embodiments and further to paragraphs [0085]-[0105] above, methods of treating or ameliorating a health condition and/or a disorder in a subject are disclosed. In some embodiments, methods of treating or ameliorating a health condition and/or a disorder in a subject include, but are not limited to, administration of an effective amount one or more ATP-competitive CaMKII inhibitor and/or pharmaceutical compositions containing one or more ATP-competitive CaMKII inhibitor as described herein. “An effective amount” as used herein can refer to a dose of ATP-competitive CaMKII inhibitor that is sufficient to confer a therapeutic effect on a subject having or suspected of having a condition, injury, disease and/or a disorder herein. In certain embodiments, a therapeutic effect for a subject having or suspected of having a disease and/or a disorder herein can include reducing the symptoms or consequences of the health condition such as brain growth and/or brain function. In some embodiments, compositions and methods disclosed herein can be administered to a subject to reduce and/or eliminate mental abnormalities during neonatal or infant development of the subject.

In some embodiments and further to paragraphs [0085]-[0106] above, methods of administering one or more ATP-competitive CaMKII inhibitor or combination therapies as disclosed herein can include placement into a subject, by a method or route that results in at least partial localization of the introduced one or more ATP-competitive CaMKII inhibitor or combination therapies at a desired site, such as the brain, such that a desired effect(s) is produced. In some embodiments, a subject can be treated with one or more ATP-competitive CaMKII inhibitor or combination therapies disclosed herein over the course of a day, for a few hours, twice daily, three times daily or more, daily, every other day, 2 times per week, weekly, every other week, monthly, or other appropriate treatment regimen. In some embodiments, a subject can be treated with a slow-release formulation where a single administration ore reduced number of administrations can last a week, a few weeks, a month or more per administration of a composition disclosed herein.

In some embodiments and further to paragraphs [0085]-[0107] above, a subject to any of the methods herein can be any subject for whom treatment or therapy is desired. In certain embodiments, a subject to any of the methods herein can be any subject having or is suspected of having a health condition in need of treatment with one or more ATP-competitive CaMKII inhibitor or combination therapies. In other embodiments, a subject to any of the methods herein can be any subject having, suspected of having or developing a brain disorder or experienced brain trauma or has reduced cognition, memory or learning disorder or any combination thereof.

In certain embodiments and further to paragraphs [0085]-[0108] above, one or more ATP-competitive CaMKII inhibitor or combination therapies and/or pharmaceutical compositions disclosed herein can be administered in dosages and by techniques well known to those skilled in the medical arts. In accordance with these embodiments, healthcare professionals will take into consideration such factors as the age, sex, weight, developmental stage and condition of the subject, and the composition form used for administration (e.g., solid vs. liquid). Dosages for humans or other mammals can be determined without undue experimentation by the skilled artisan, from this disclosure, and the knowledge in the art.

In some embodiments and further to paragraphs [0085]-[0109] above, kits are provided for use in treating or alleviating a targeted disease or condition treatable by use of one or more ATP-competitive CaMKII inhibitor or combination therapies disclosed herein. In some embodiments, kits can include instructions for use in accordance with any of the methods described herein. Instructions can include a description of administration of any one or more ATP-competitive CaMKII inhibitor or combination therapies and/or pharmaceutical compositions described herein and optionally one or more additional therapies to treat, delay the onset, or alleviate a target disease (e.g., AD) as those described herein. In some embodiments, kits disclosed herein can further include a description of selecting an individual suitable for treatment based on identifying whether that individual has the target disease, e.g., applying one or more diagnostic methods. In some embodiments, the instructions can include a description of administering one or more ATP-competitive CaMKII inhibitor or combination therapies to a subject at risk of or having the target disease or health condition. In other embodiments, kits can include instructions for assessing success of a treatment disclosed herein such as testing cognition or memory in a treated subject and adjusting an administered composition disclosed herein based on outcome of the tests such as increasing or decreasing dose and/or frequency of administration.

In certain embodiments and further to paragraphs [0085]-[0110] above, a label or package insert herein can indicate that the composition is used for treating, delaying the onset and/or alleviating a health condition (e.g., cognitive condition or other brain disorder). Instructions can be provided for practicing any of the methods described herein.

In certain embodiments and further to paragraphs [0085]-[0111] above, kits disclosed herein can further include suitable packaging. Suitable packaging includes, but is not limited to, syringes, vials, bottles, jars, flexible packaging (e.g., sealed Mylar or plastic bags), and the like. Also contemplated herein are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle).

### EXAMPLES

The following examples are included to illustrate certain embodiments. It should be appreciated by those of skill in the art that the techniques disclosed in the examples which follow represent techniques discovered to function in the practice of the claimed methods, compositions, and apparatus. However, those of skill in the art should, in light of the present disclosure, appreciate that changes can be made to certain examples or some embodiments which are disclosed and still obtain a like or similar result without departing from the spirit and scope of the invention.

The following examples describe a series of experiments conducted to determine the relative contributions of CaMKII enzymatic activity versus GluN2B binding in LTP induction and other observations affecting brain function. Historically, it has been thought that CaMKII activity cannot be blocked without interfering with LTP induction. Surprisingly, the instant inventions provide inhibitors that specifically target CaMKII adverse activities without interfering with CaMKII binding of GluN2B of NMDAR and preserve and/or restore LTP induction.

### Example 1

Photo-Activation of paCaMKII Induces GluN2B Binding

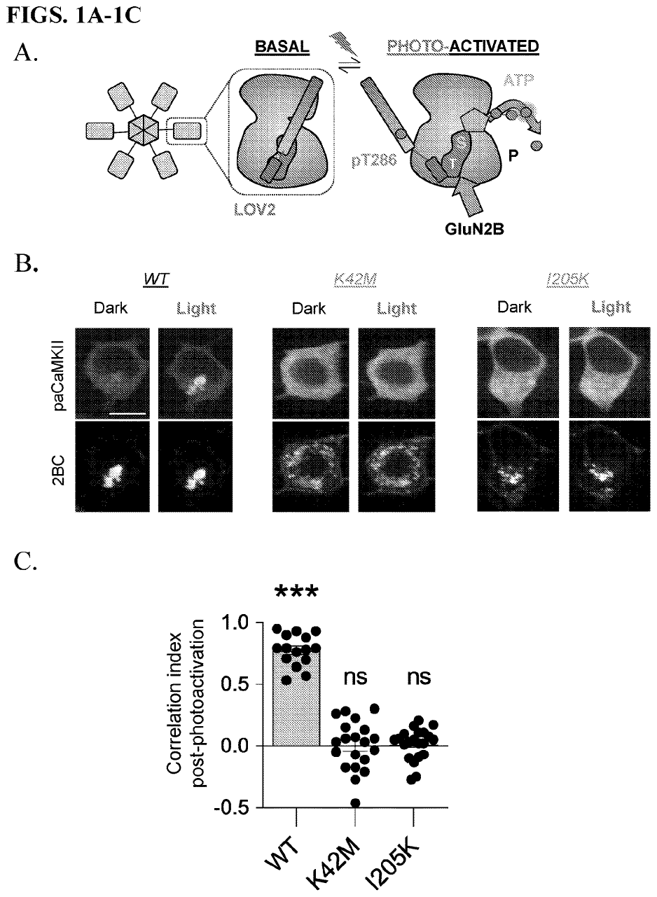

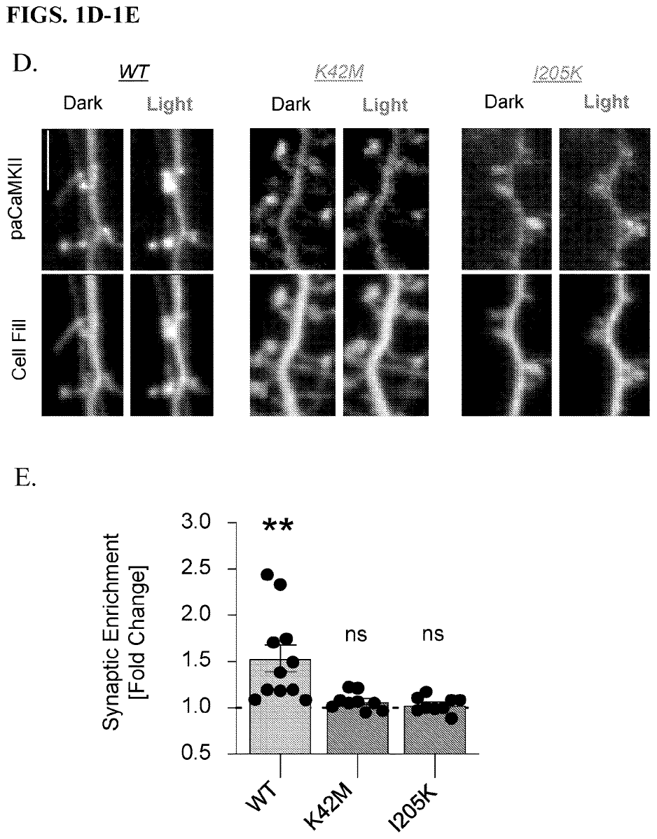

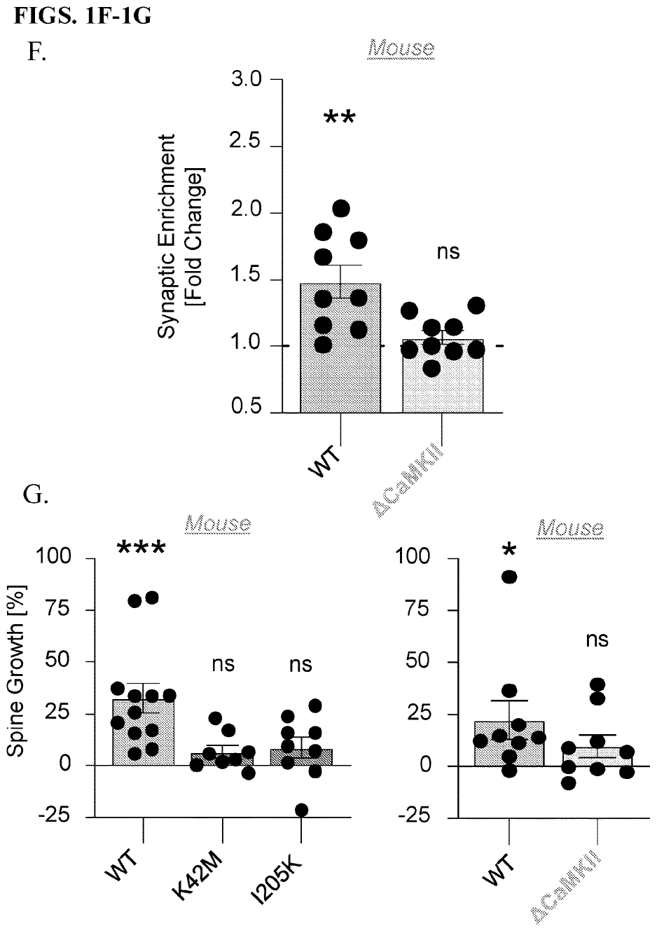

Photoactivatable CaMKII (paCaMKII) is a fusion protein comprised of a light sensitive domain (LOV2) fused to CaMKIIα as is described in Shibtata et al., (Photoactivatable CaMKII induces synaptic plasticity in single synapses Nat Commun 12, 751 (2021). doi.org/10.1038/s41467-021-21025-6, which is incorporated herein by reference in its entirety). It has been demonstrated that direct photoactivation of paCaMKII is sufficient to induce structural LTP in hippocampal neurons even in the absence of neuronal stimulation, which suggests that CaMKII enzymatic activity is sufficient for LTP induction. However, photo-activation of paCaMKII involves displacement of the autoinhibitory domain to expose not only the substrate binding surface (S-site) but also the neighboring GluN2B-binding surface (T-site) (see FIG. 1A). This is similar to what occurs when CaMKII is activated normally (e.g., via stimulation by Ca²⁺/CaM). It is also noted that these two sites (S-site and T-site) form a continuous groove and can act together in protein binding, with GluN2B binding progressing from an initial S-site binding to a more stable T-site binding mode as demonstrated in FIG. 1A. The CaMKII binding site on GluN2B around S1303 shares homology with the CaMKII regulatory domain region around T286, and the T-site binding mode is exemplified by the T286 interaction with the kinase domain in the basal state whereas the S-site binding mode is exemplified by the T286 interaction during its autophosphorylation by a neighboring kinase subunit within the CaMKII holoenzyme). Thus, it was hypothesized that photoactivation of paCaMKII may additionally directly promote binding to GluN2B (FIG. 1A).

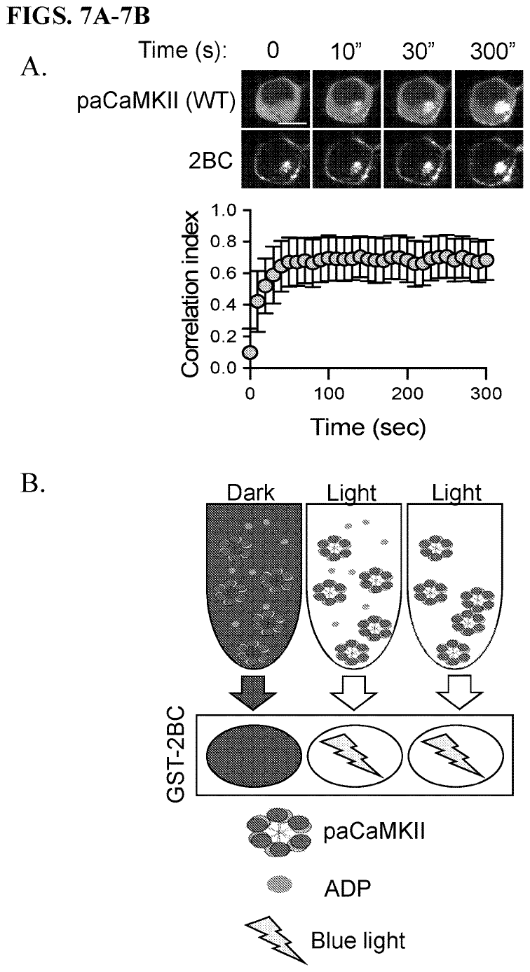

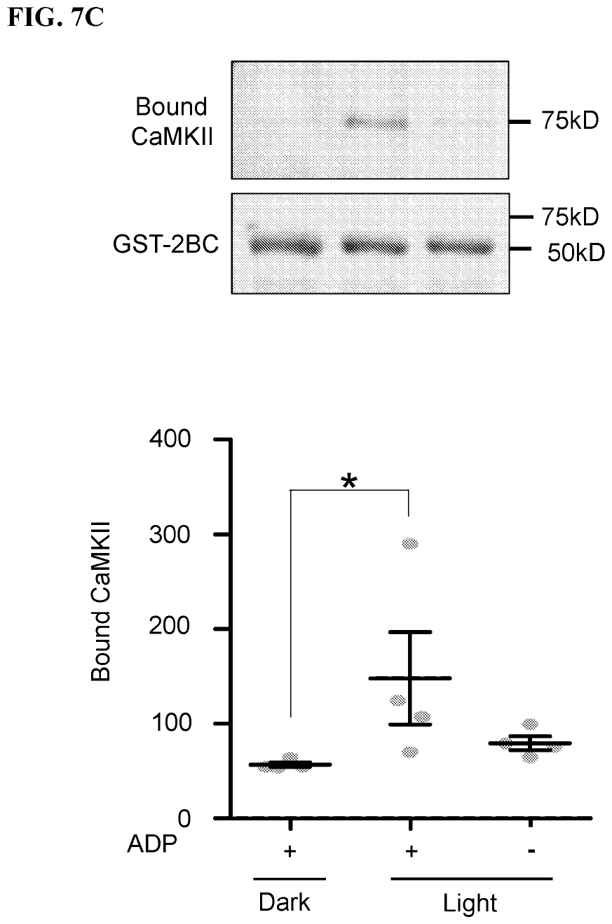

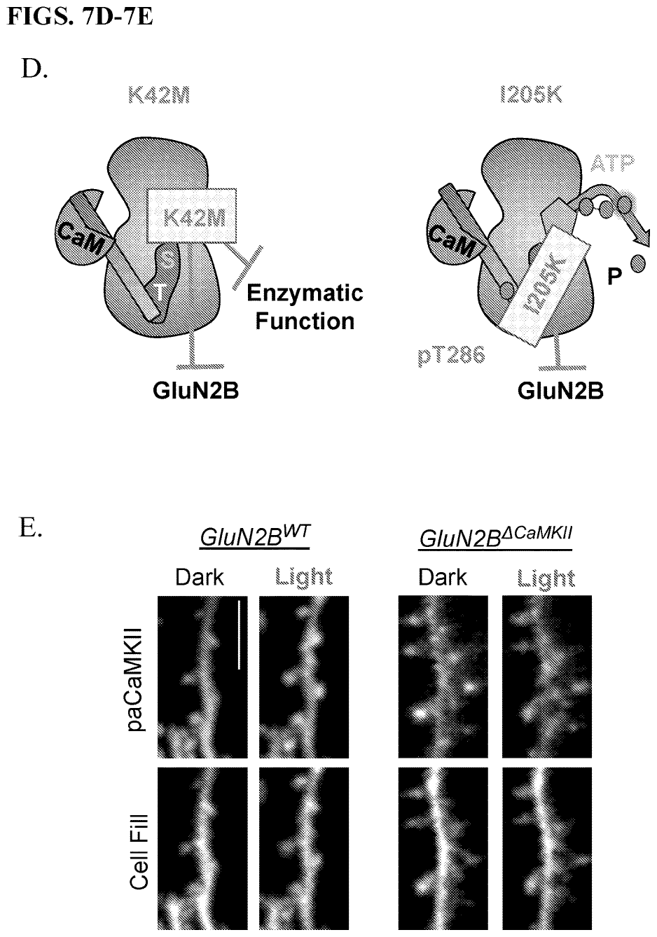

To test this, in a first exemplary method, co-expression of GFP-paCaMKII and mCh-GluN2B C-tail was performed in HEK293 cells and monitored for colocalization before and after blue-light stimulation. FIG. 1B demonstrates illustrative confocal microscopy images of these HEK293 cells co-expressing GFP-paCaMKII (WT, K42M, or I205K) and pDisplay-mCherry-GluN2B-c-tail (mCh-2BC) before and after photoactivation and FIG. 7A demonstrates a time-course of correlation indices of GFP-paCaMKII and mCh-2B after photoactivation. Surprisingly, it was found that photoactivation did induce robust colocalization of GFP-paCaMKII and mCh-GluN2B C-tail (FIG. 1A-1B and FIG. 7A) indicating that photoactivation of paCaMKII indeed also promotes GluN2B binding. This light-induced binding of paCaMKII to the GluN2B C-tail was also corroborated by an in vitro binding assay. Specifically, as diagrammed in FIG. 7B, an exemplary experiment was performed to use blue light to induce paCaMKII pulldown of immobilized GST-2BC. FIG. 7C demonstrates that binding of CaMKII-F89G to immobilized GST-2BC was induced by blue light in the presence of either 100 μM ADP alone or without a nucleotide.

Additional exemplary methods were performed to further validate the finding that CaMKII activation leads to GluN2B binding and that this contributes to LTP. In one exemplary method, two mutations that render CaMKII unable to bind GluN2B were introduced into the paCaMKII construct: the mutation of the CaMKII T-site (I205K) or the CaMKII ATP-binding site (K42M). Neither of these mutant paCaMKII constructs demonstrated colocalization with mCh-GluN2B after photoactivation in HEK cells (FIG. 1C) which aligns with the effect of these mutations on CaMKII activity (graphically depicted in FIG. 7D). Meanwhile, in rat hippocampal neurons, regulated GluN2B binding is needed for normal LTP and is responsible for LTP-stimuli inducing movement of CaMKII to dendritic spines, the post-synaptic compartments of excitatory synapses. Accordingly, in a second exemplary method, dissociated rat hippocampal cultures were transduced to express GFP-paCaMKII (WT, K42M, or I205K) and mCherry throughout the cytoplasm. This allowed the visualization of co-localization of paCaMKII to the synapse (labeled with mCherry) after photoactivation of paCaMKII. It was found that photoactivation of GFP-paCaMKII directly induced its movement to synapses, even in the absence of any other stimulation and this synaptic movement of GFP-paCaMKII was completely abolished by two distinct CaMKII mutations, I205K and K42M which both inhibit CaMKII binding to GluN2B (FIG. 1D and FIG. 1E). Finally, in another exemplary method, this co-localization experiment was repeated using hippocampal cells obtained from mice having a mutation in GluN2B that prevents CaMKII binding (GluN2B^{ΔCaMKII}). It was found that expression of this GluN2B mutant also abolished synaptic localization of CaMKII after photoactivation of GFP-paCaMKII (FIG. 1F and FIG. 7E). These results together indicate that photoactivation causes the same type of synaptic enrichment of CaMKII as LTP stimuli.

### Example 2

paCaMKII Requires GluN2B Binding for sLTP

In this example, effect of CaMKII association with GluN2B on spine growth during LTP was assessed. Specifically, in one exemplary method, whether spine growth after photoactivation of paCaMKII required GluN2B binding was examined. Spine growth is considered by one of skill in the art as a readout for structural LTP (sLTP) and was monitored here in live neurons using mCherry as cell fill (see Materials and Methods). Photoactivation of GFP-paCaMKII wildtype induced robust spine growth in rat hippocampal neurons and this effect was blocked by the I205K and K42M mutations that inhibit GluN2B binding (FIG. 1D and FIG. 1G). Interestingly, while the K42M mutation also abolishes enzymatic CaMKII activity, the I205K mutation does not, indicating that a structural function such as GluN2B binding is required for photoactivation-induced spine growth and that enzymatic activity of paCaMKII alone is not sufficient for this induction. In further exemplary methods, photoactivation-induced spine growth was also observed in hippocampal neurons from WT mice, but not in cultures from mice with the GluN2B^{ΔCaMKII} mutation that prevents CaMKII binding (FIG. 1G and FIG. 7E).

### Example 3

Identification of AS283 and AS105 as Selective Inhibitors of CaMKII Activity that do not Interfere with GluN2B Binding

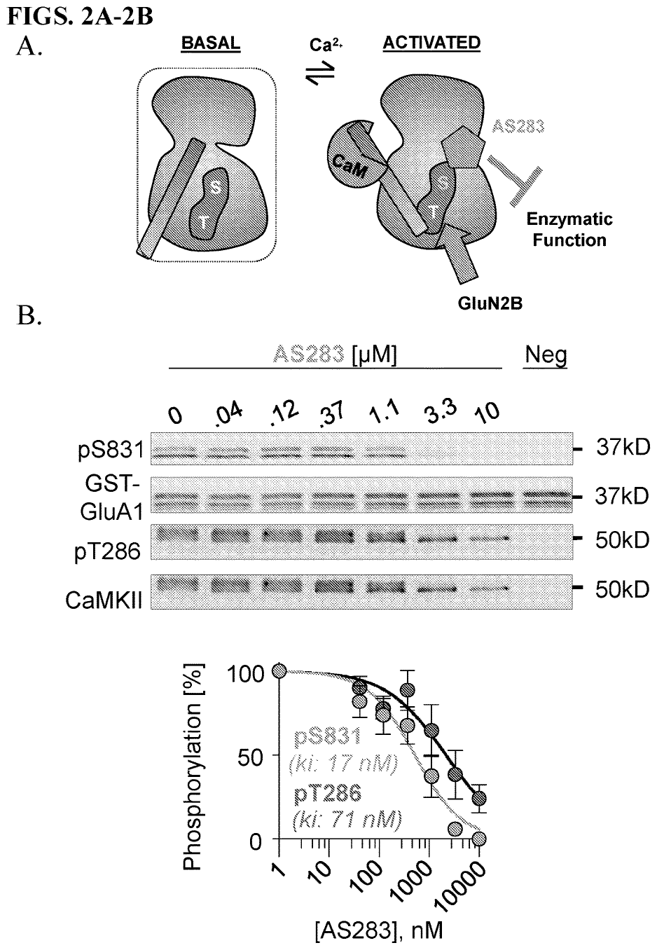

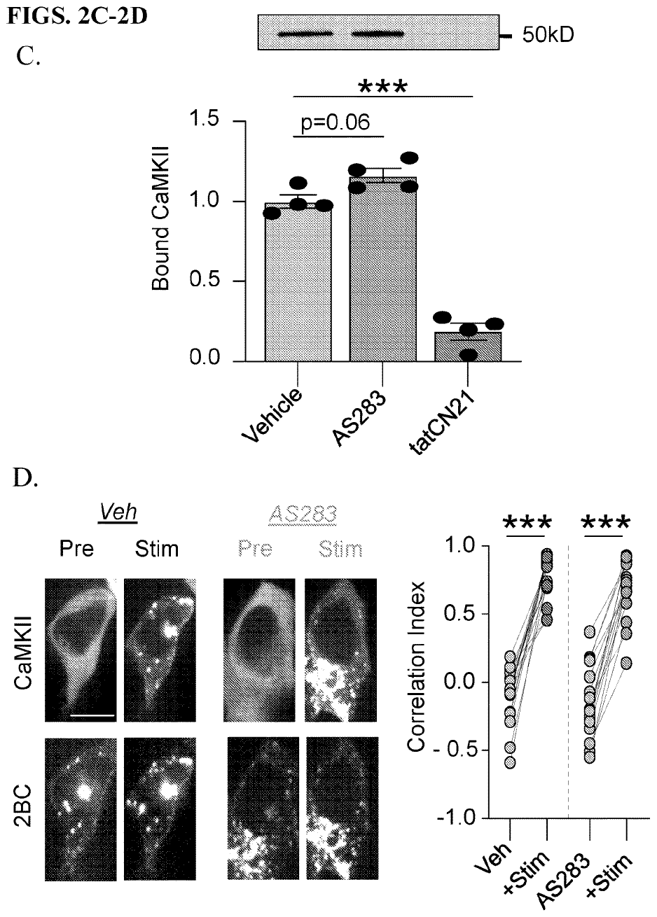

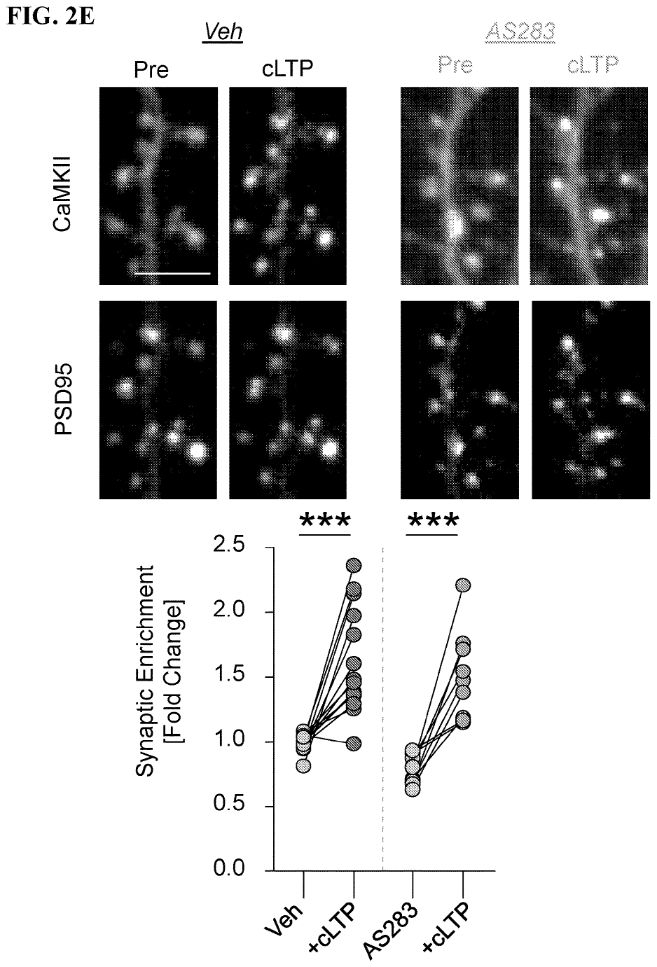

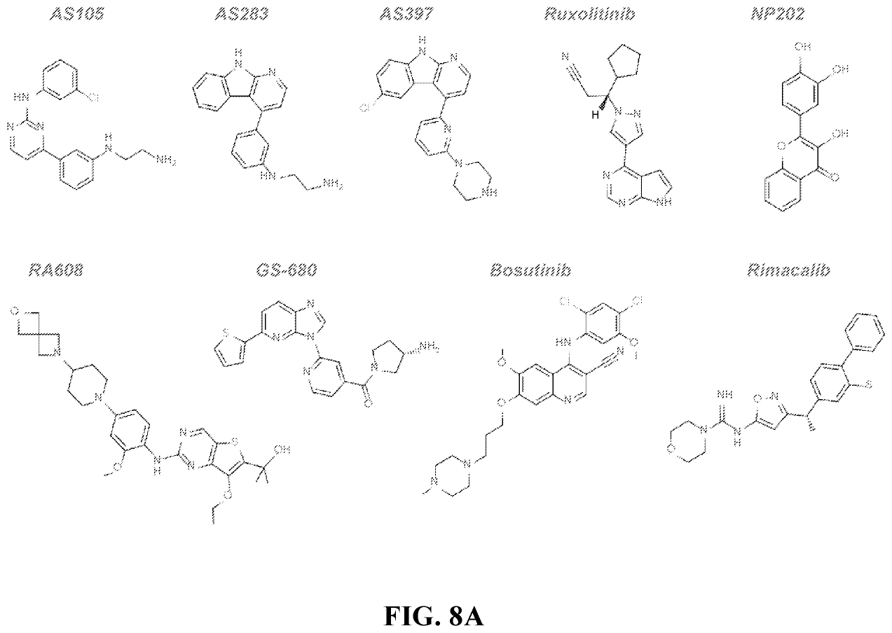

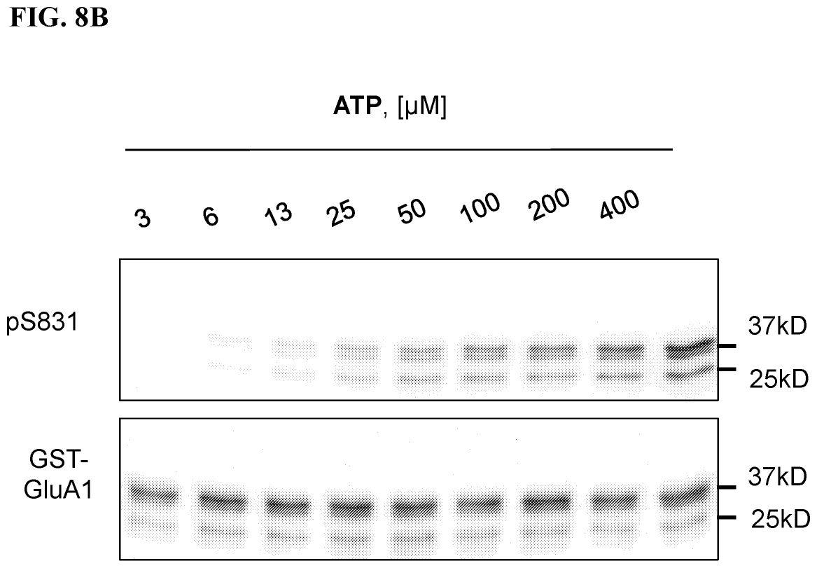

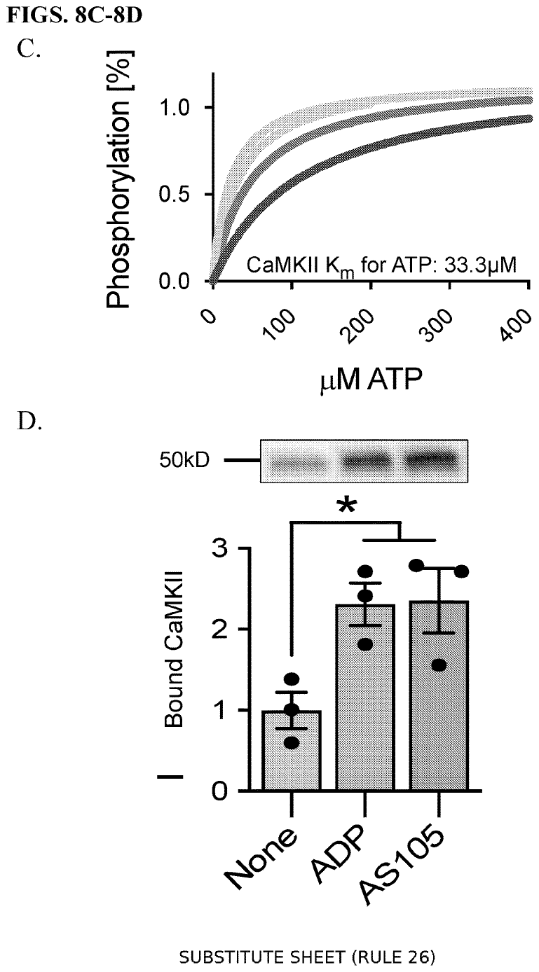

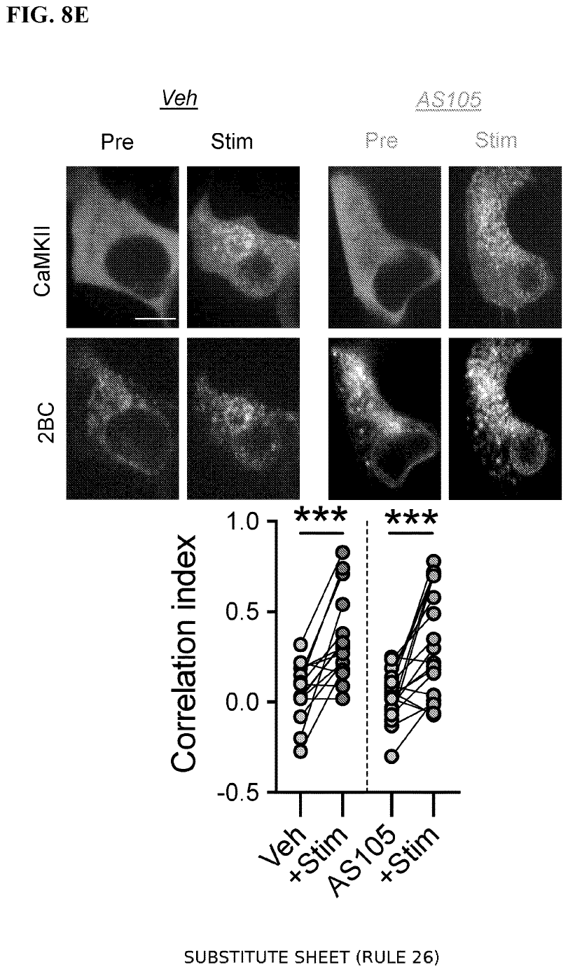

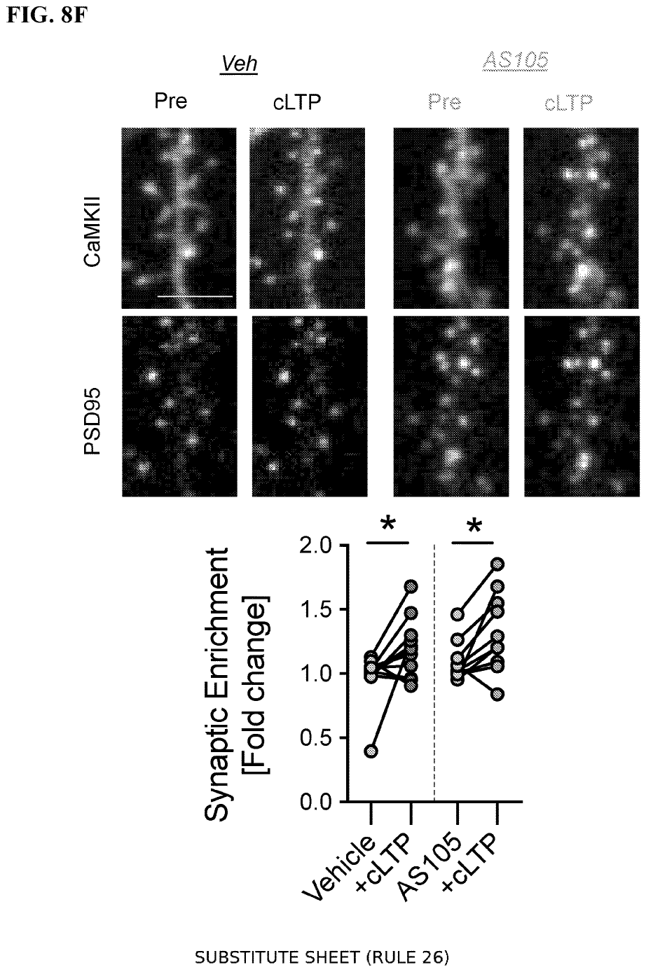

Normal GluN2B binding by CaMKII requires stimulation of CaMKII by Ca²⁺/CaM as well as occupation of its ATP-binding pocket (FIG. 2A). However, the ATP binding pocket can be occupied by either ATP, ADP, or ATP-competitive inhibitors such as the broad-spectrum kinase inhibitors staurosporine or H7. In a first exemplary method, whether new ATP-competitive and CaMKII-selective inhibitors, AS283 and AS105, illustrated in FIG. 8A, behave similarly was tested and confirmed.

In a series of exemplary methods, an in vitro kinase assay was used where purified CaMKIIα is incubated at 30° C. with GST-GluA1 alongside ATP. CaMKIIα normally phosphorylates S831 on the GluA1 subunit and this phosphorylation can be measured as a readout of CaMKIIα enzymatic activity (see e.g., FIG. 8B). Performing this in vitro kinase assay in increasing concentrations of ATP generated a Km value of ATP (to activate CaMKII) of 33.3 μM, which was derived from the concentration of ATP that achieved a half-maximal response for four experiments (FIG. 8B-FIG. 8C). In another exemplary method, binding of purified CaMKIIα to immobilized GST-GluN2B-c was induced by Ca²⁺/CaM in the presence of a vehicle (none), 4 mM ADP or with 10 AM AS105. It was found that both ADP and AS105 induced binding (FIG. 8C). At the same time, two exemplary methods were performed to observe ionomycin or chemical LTP (cLTP) induced co-localization of CaMKII and GluN2B either in HEK293 cells co-expressing GFP-CaMKII and mCherry-2BC or dissociated rat hippocampal cultures expressing GFP-CaMKII and mCh-PSD95 intrabody. HEK293 cells, incubating with vehicle or 10 μM AS105, were subjected to ionomycin. Hippocampal cultures, also incubating with either vehicle or 10 μM AS105, were treated with a chemical LTP (cLTP) stimulus (e.g., 100 μM glutamate, 10 μM glycine for 45 seconds). Confocal microscopy images were taken before and after each stimulus. As is illustrated in FIGS. 8D-8E AS105 did not interfere with ionomycin induced or illustrated in LTP induced co-localization of CaMKII and GluN2B in either cell type. Together, these experiments demonstrated that AS105 inhibits the enzymatic activity of CaMKII without inhibiting either GluN2B binding or stimulation-induced movement to excitatory synapses (FIG. 8B-8F).

AS283 is another inhibitor having even more CaMKII-selectivity compared to AS105, as demonstrated in the Table below. In this table, residual kinase activity in the presence of 1 μM of each inhibitor is illustrated as a percentage of maximal activity. As seen in the table, AS283 is more selective at inhibiting CaMKII specifically compared to AS105. Also notably, the one remaining strongly cross-inhibited kinase (Flt3) is not significantly expressed in hippocampus. Two other kinases (KDR and p70S6K) remain somewhat inhibited by AS283, but for a block comparable to CaMKII activity, higher concentrations could be needed.

| | AS105/Scios | AS283/Tricyclic |
|---|---|---|
| Arg(h) | 87 | 96 |
| AMPKα1(h) | 38 | 70 |
| Aurora-C(h) | 57 | 63 |
| CaMKI(h) | 83 | 107 |
| CaMKIIβ(h) | 6 | 5 |
| CaMKIIγ(h) | 2 | 2 |
| CaMKIIδ(h) | 2 | 2 |
| CaMKIIα(h) | | 3 |
| CaMKIV(h) | 101 | 90 |
| CDK2/cyclinE(h) | 2 | 41 |
| CDK5/p35(h) | 1 | 33 |
| cKit(h) | 91 | 50 |
| FAK(h) | 75 | 68 |
| FGFR1(h) | 91 | 58 |
| Flt3(h) | 2 | 3 |
| GSK3α(h) | 6 | 82 |
| GSK3β(h) | 11 | 87 |
| IGF-1R(h) | 121 | 110 |
| JAK2(h) | 134 | 121 |
| JAK3(h) | 89 | 92 |
| JNK2α2(h) | 79 | 82 |
| KDR(h) | 28 | 15 |
| MAPK1(h) | 105 | 102 |
| MAPK2(h) | 106 | 97 |
| MEK1(h) | 111 | 85 |
| mTOR(h) | 102 | 115 |
| p70S6K(h) | 77 | 18 |
| PAK2(h) | 104 | 76 |
| PDGFRβ(h) | 80 | 111 |
| PDK1(h) | 109 | 78 |
| Pim-1(h) | 69 | 50 |
| Pim-3(h) | 109 | 97 |
| PKBγ(h) | 76 | 42 |
| PKCα(h) | 51 | 49 |
| PKCδ(h) | 73 | 39 |
| PKG1α(h) | 61 | 48 |
| PKG1β(h) | 68 | 38 |
| Ret(h) | 55 | 44 |
| ROCK-II(h) | 43 | 77 |
| SAPK2a(h) | 99 | 95 |
| SGK(h) | 97 | 86 |
| PI3 Kinase | 96 | 100 |
| (p110a/p85a)(h) | | |

Therefore, in another exemplary method, the ability of AS283 to selective inhibit CAMKII enzymatic activity without interfering with its binding to GluN2B was tested. In a first method, in vitro kinase reactions were performed at 30° C. in 1 mM ATP measuring purified CaMKIIα phosphorylation of GST-GluA1 S831 with varying concentrations of AS283 (0.41-10 μM). It was found that AS283 effectively inhibited both phosphorylation of GluA1 S831 (pS831) and autophosphorylation of T286 (pT286) in these experiments, although the exogenous pS831 appeared to be inhibited slightly more potently than the pT286 that occurs within the holoenzyme, with apparent Ki's of 17 nM and 71 nM, respectively (FIG. 2B). To calculate the Ki's, a CaMKII Km for ATP of 33.3 μM was used as calculated in FIG. 7C.

In another exemplary method, binding of purified CaMKIIα to immobilized GST-GluN2B-c was induced by Ca²⁺/CaM in the presence of 1 mM ADP alone or with 10 AM AS283 or 5 μM tatCN21 (a non-ATP competitive CaMKII inhibitor). In further exemplary methods, the effect of AS283 on co-localization of GFP-CaMKII and mCherry-BC was observed in HEK293 cells or GFP-CaMKII and mCh-PSD95 in hippocampal neurons after ionomycin stimulation in HEK293 cells or chemical LTP stimulation in neurons (cLTP, 100 μM glutamate, 10 μM glycine; 45 seconds). Importantly, and in contrast to tatCN21 (a competitive CaMKII inhibitor that binds to the GluN2B binding site) (5 μM), AS283 (10 μM) did not inhibit CaMKII binding to GluN2B in these in vitro binding assays in the presence of ADP (FIG. 2C). Likewise, AS283 failed to inhibit CaMKII binding to GluN2B in the co-localization assays in HEK cells (FIG. 2D) or in neurons in response to chemical LTP stimuli (FIG. 2E).

This Example demonstrates that AS105 and AS283 are useful tools that inhibit enzymatic activity of CaMKII with high potency and selectivity without disrupting its binding to GluN2B. Therefore, they can be used to distinguish between enzymatic and structural functions of CaMKII.

### Example 4

AS283 does not Block LTP Induction

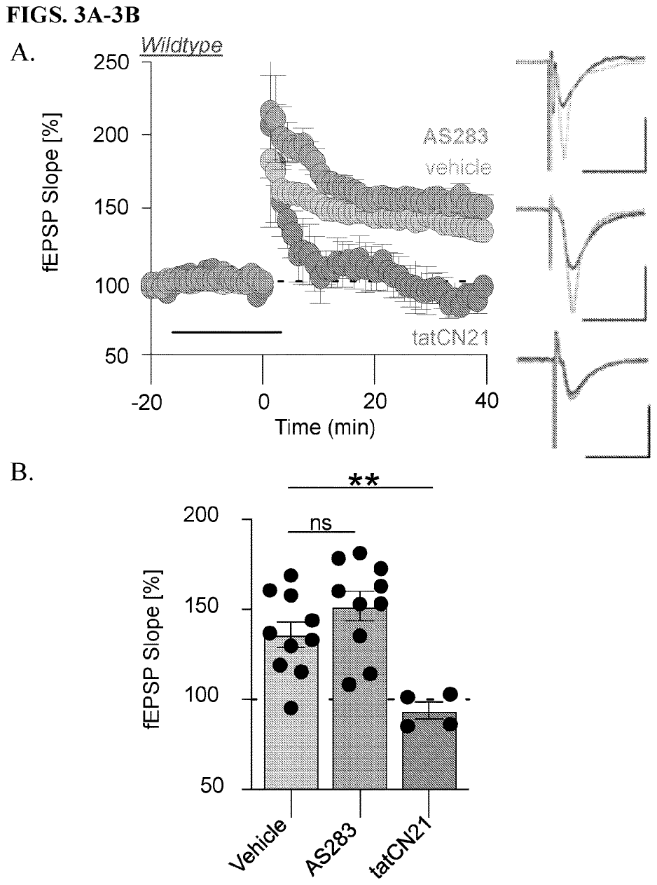

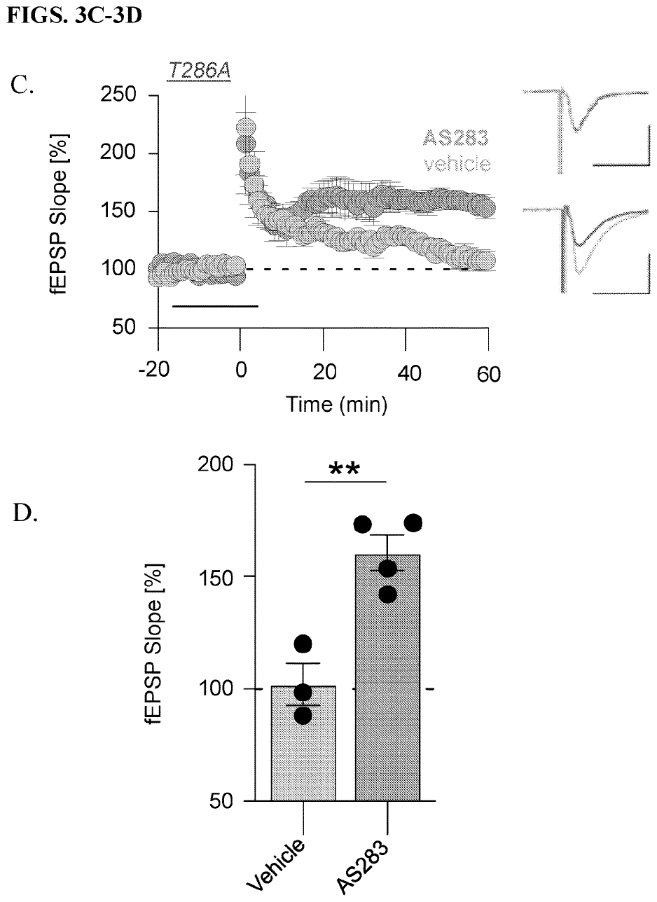

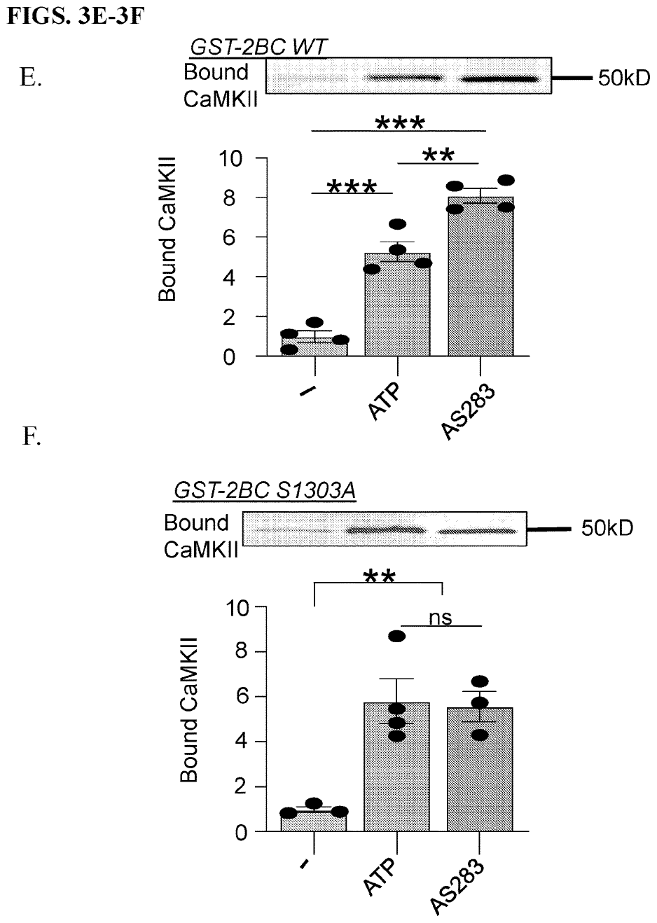

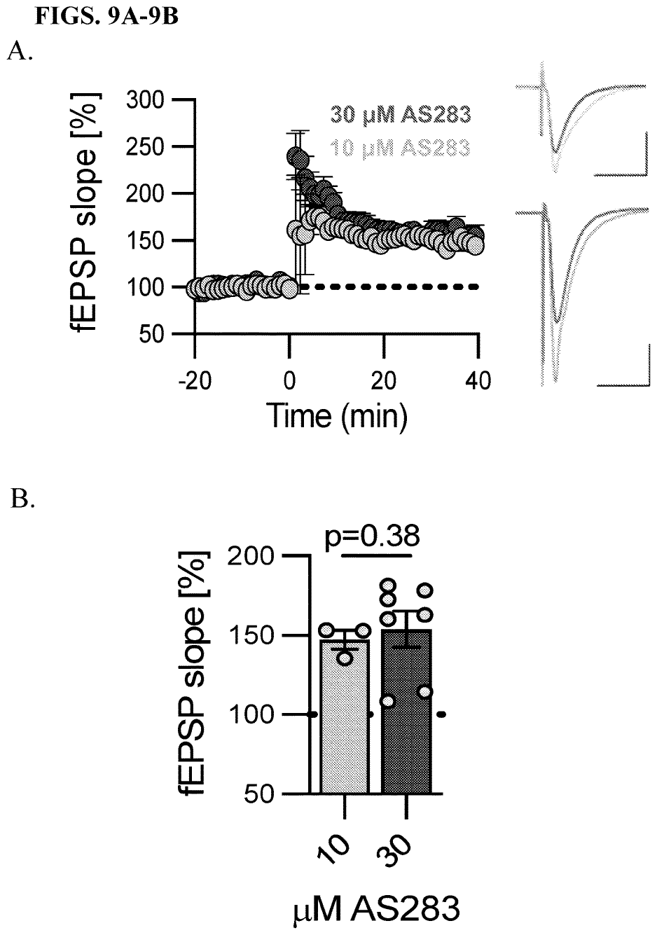

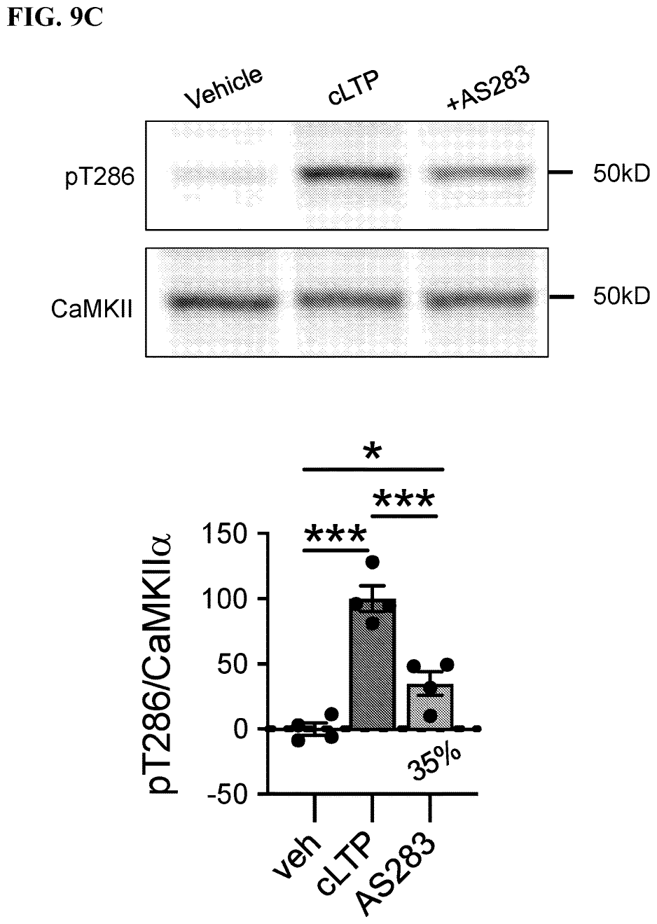

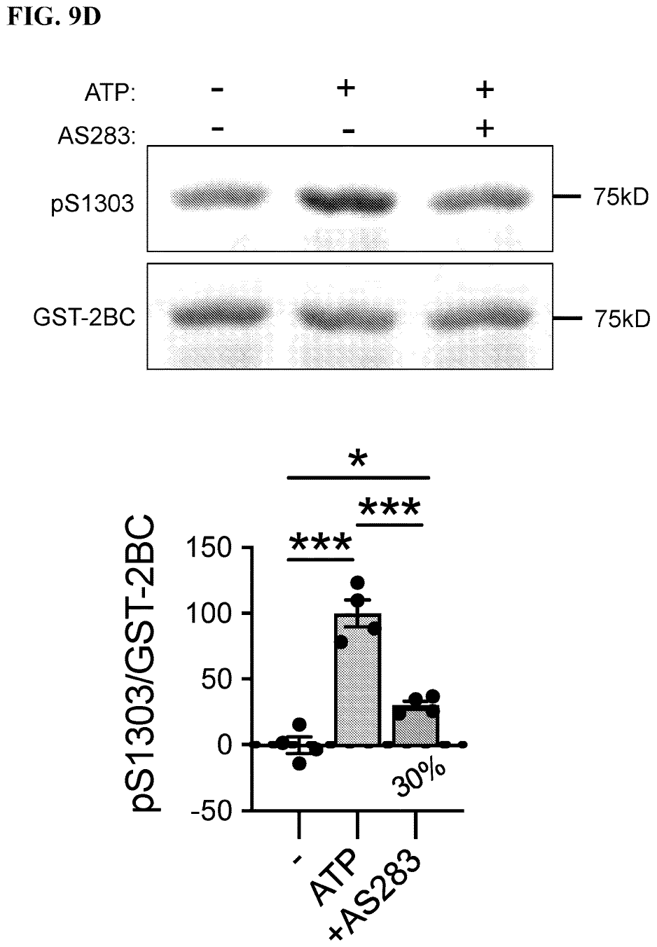

In another exemplary method, the effect of AS283 (established as an effective CaMKII inhibitor that does not block binding to GluN2B) on LTP induction at the hippocampal CA3 to CA1 synapse in wildtype mice was tested using field recordings in acute slices. Long term potentiation (LTP) is induced by high frequency stimulation (HFS; 2×100 Hz. 10 sec interval, see Materials and Methods) of CA3-CA1 Schaffer collateral pathways in WT mice and is observed by an increase in slope of field excitatory post synaptic potentials (fEPSPs) that persists after the stimulus is removed. When testing the effect of certain agents on LTP induction (i.e., AS283 or tatCN21), slices were incubated with the agents for 15 minutes before LTP induction and washed out 5 minutes after LTP induction. Under these experimental conditions, LTP was effectively blocked by tatCN21 (5 μM; FIGS. 3A-3B), as expected from previous studies. Previous studies have also demonstrated that LTP inhibition can be achieved with broad-spectrum ATP-competitive inhibitors such as H7. By contrast, incubation with the CaMKII-selective AS283 (10 or 30 μM) failed to block LTP induction (FIGS. 3A-3B and FIGS. 9A-9B). This result was highly surprising, because at least one enzymatic CaMKII reaction is thought to be needed for LTP induction: autophosphorylation of CaMKII at T286. This suggested three possibilities: (i) AS283 is not effective at inhibiting pT286, (ii) AS283 is not effective in hippocampal slices, or (iii) AS283 somehow circumvents a need for pT286. Somewhat surprisingly, the following Examples provide strong and convincing support for the latter, i.e. that AS283 effectively circumvents the need of pT286 in LTP at CA1 synapses.

### Example 5

### AS283 Restores LTP in T286A Mutant Mice

While AS283 also inhibited pT286 with slightly less potency than an exogenous substrate, this inhibition was still very potent and effective, at least in vitro (see FIG. 2A) and in hippocampal cultures where AS283 specifically blocked increase in pT286 levels observed after chemical LTP stimulation (FIG. 9C). In this Example, other possibilities of AS283 action were tested. If AS283 blocks pT286 but not LTP because it also circumvents the need of pT286 for LTP, then AS283 should not only fail to block LTP in WT mice (as illustrated in FIGS. 3A-3B) but should also restore the capacity for LTP induction in T286A mutant mice. Surprisingly, this was exactly what was observed experimentally. Specifically, in an exemplary method, the addition of AS283 (10 μM) enabled LTP in slices from the T286A mutant mice (FIGS. 3C-3D). This result directly demonstrates the effectiveness of AS283 in slices and strongly supports the notion that LTP induction needs structural CaMKII functions rather than its enzymatic activity towards exogenous substrates.

### Example 6

### AS283 Enhances CaMKII Binding to GluN2B

One function of pT286 in LTP is enhancing GluN2B binding. Therefore, another exemplary method is described herein testing the possibility AS283 may circumvent pT286 function by directly enhancing GluN2B binding even in absence of pT286. In this exemplary method, binding of purified CaMKIIα to immobilized GST-GluN2B-c (WT or S1303A), induced by Ca²⁺/CaM was tested in three different conditions: without nucleotide, with 1 mM ATP or with 10 M AS283. It was found that when ATP was substituted with AS283, Ca²⁺/CaM-induced CaMKII binding to immobilized WT GluN2B was significantly enhanced, even though this prevents the phosphorylation of T286 (FIG. 3E). Thus, even though both ATP and pT286 enhance CaMKII binding to GluN2B, AS283 can directly enhance this binding even more. As a consequence, in the presence of AS283, pT286 is dispensable for proper GluN2B binding and LTP.

One possible mechanism by which AS283 could enhance GluN2B binding more than ATP is by suppressing CaMKII-mediated phosphorylation of GluN2B at S1303, as this phosphorylation within the CaMKII binding site on GluN2B reduces CaMKII binding. This was demonstrated in another exemplary method where an identical in vitro binding experiment was performed using a phosphorylation-incompetent GluN2B S1303A mutant. It was found that the enhancing effects of AS283 and ATP on CaMKII binding were indistinguishable (FIG. 3F). Furthermore, for the wild type GluN2B C-terminus, AS283 inhibited the CaMKII-mediated S1303 phosphorylation (FIG. 9D), as expected.

### Example 7

### CaMKII F89G Mutation as a Powerful Tool

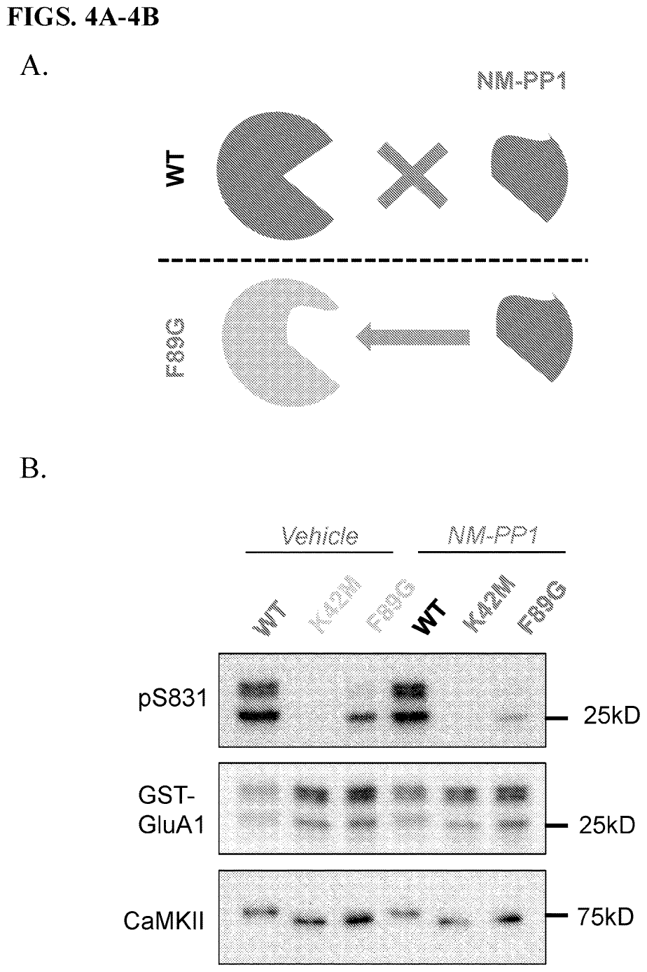

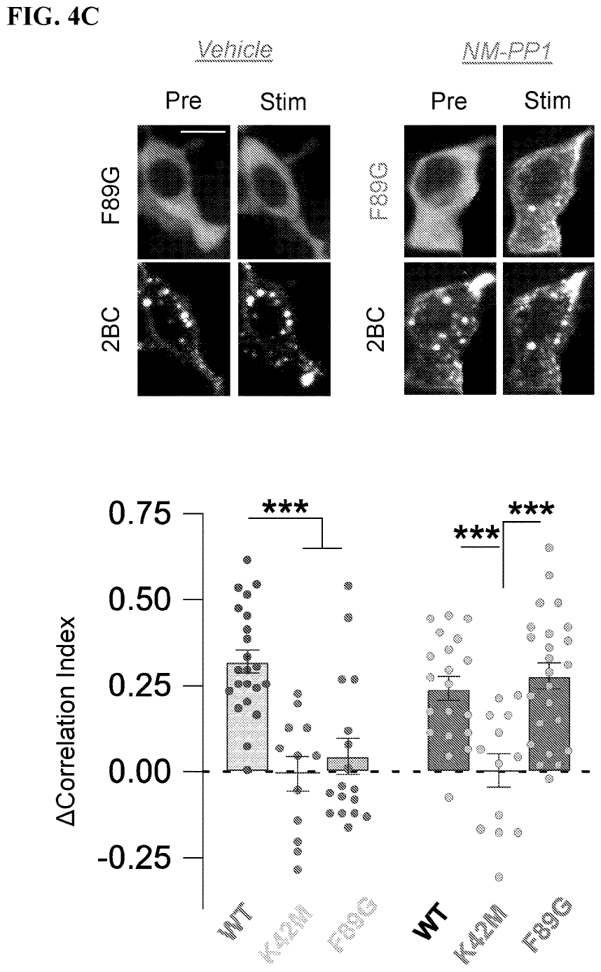

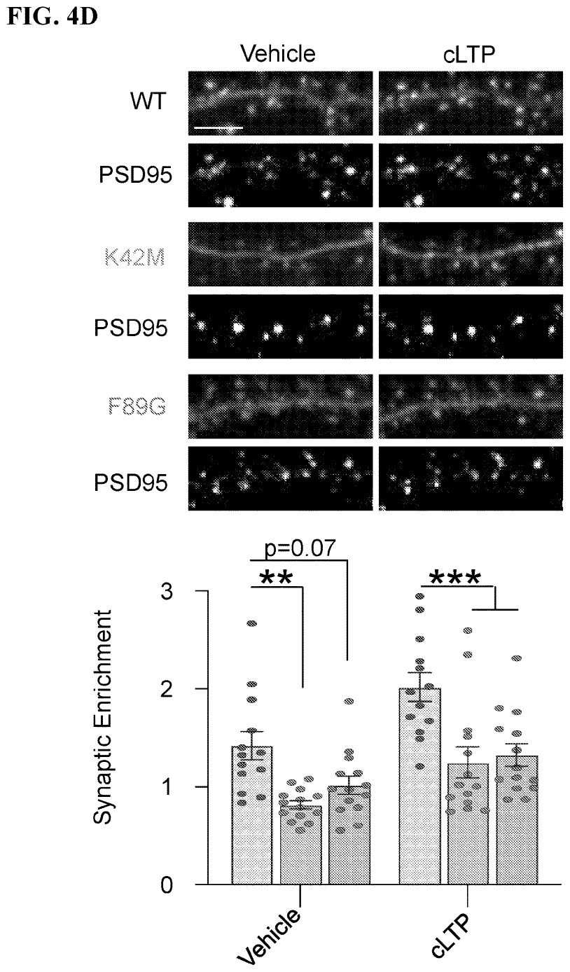

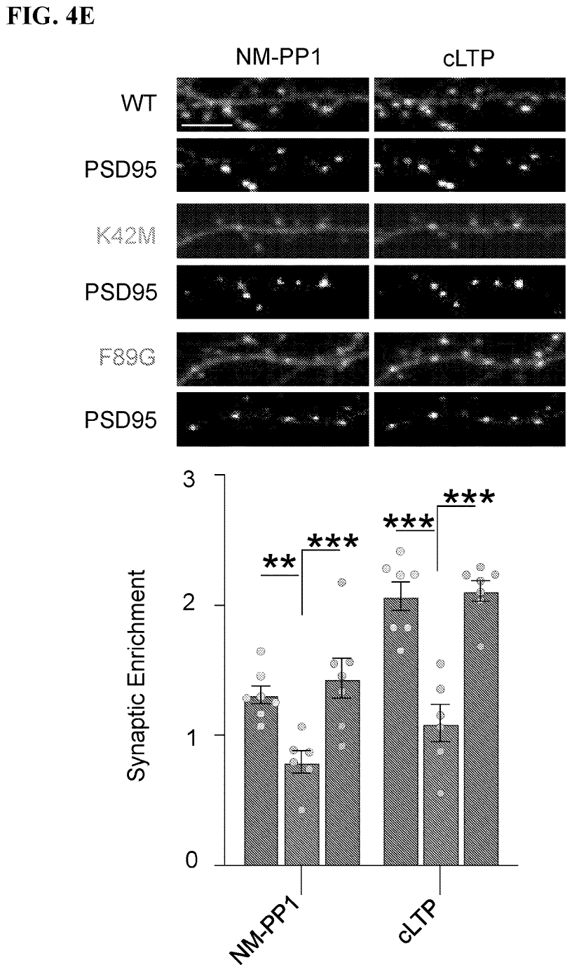

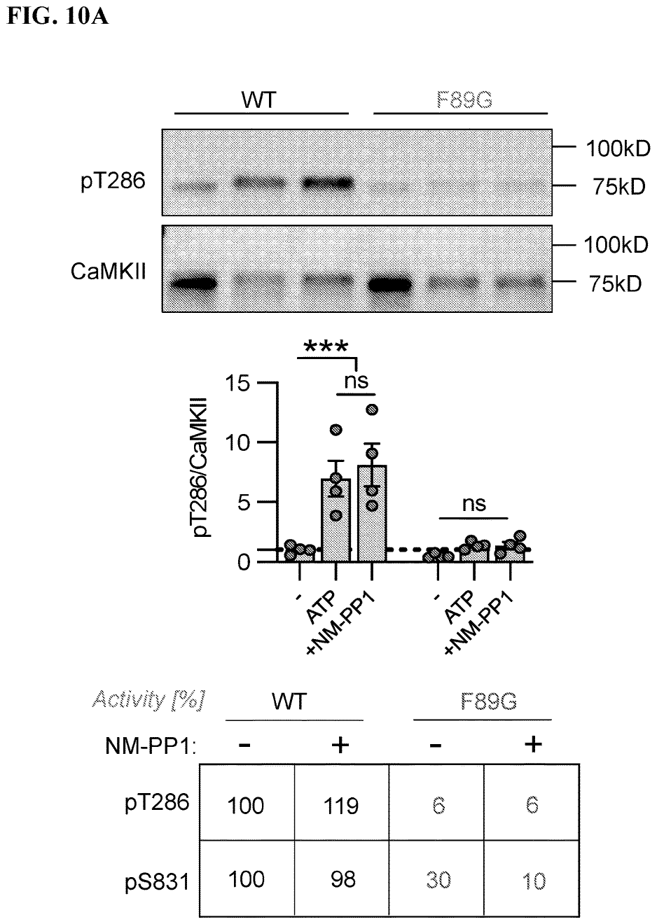

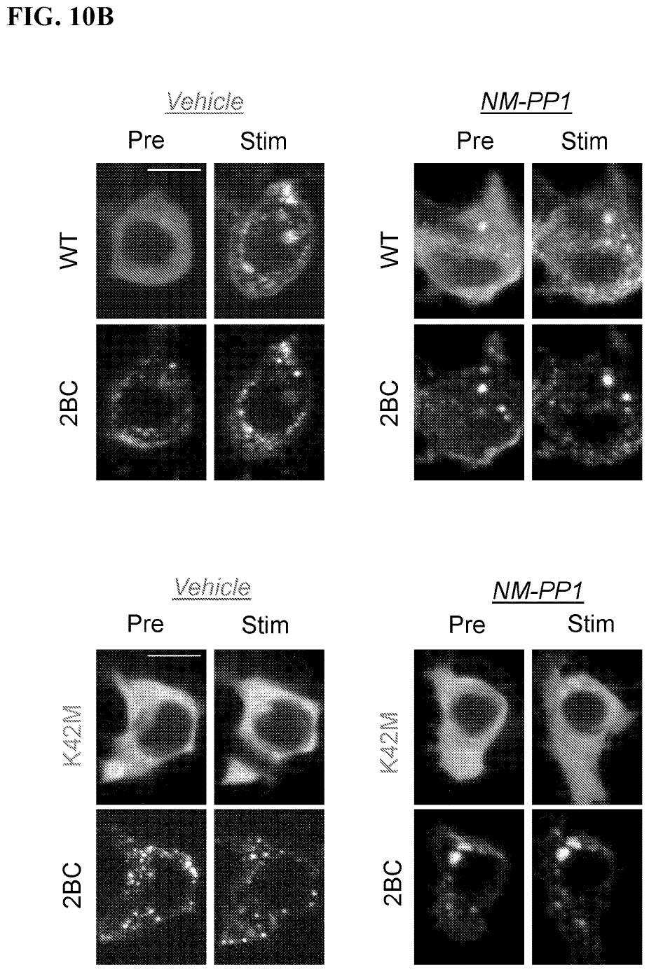

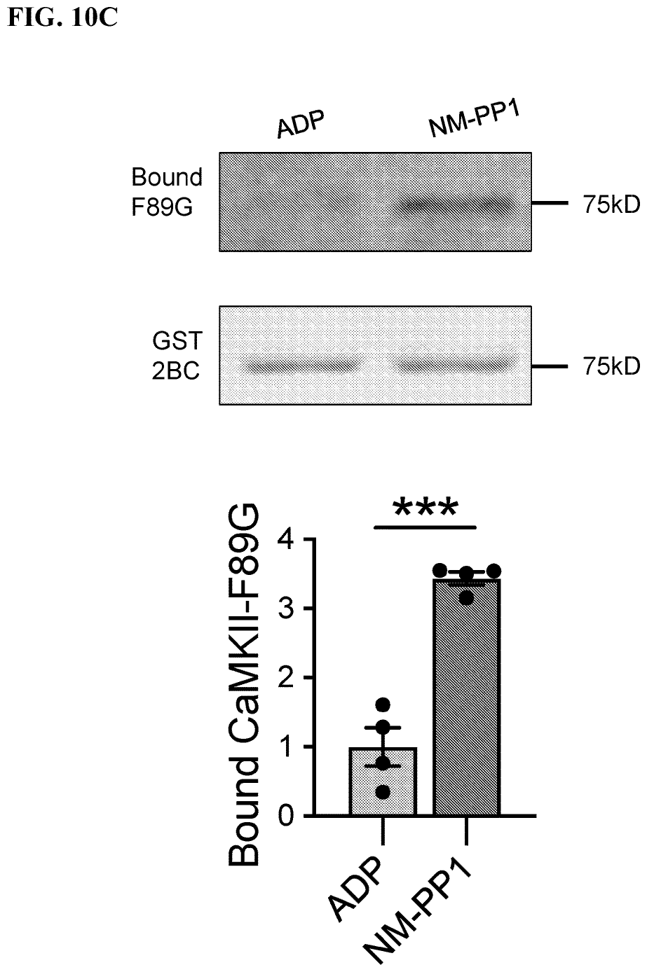

AS283 is highly selective for CaMKII (see the Table in Example 3, above) and the pharmacogenetic restoration of the capacity for LTP induction in T286A mice by AS283 provided powerful and convincing support for the conclusion that structural rather than enzymatic functions of CaMKII are essential for LTP induction. In this Example, further exemplary methods are described to further support this conclusion using a pharmacogenetic approach with the CaMKII F89G “Shokat” mutation, which enlarges the ATP binding pocket to enable selective ATP-competitive inhibition with NM-PP1, an inhibitor that does not affect wildtype kinases (FIG. 4A). Surprisingly, the F89G mutation also directly reduced CaMKII enzymatic activity measured using in vitro kinase assays as described above (FIG. 4B and FIG. 10A); however, any remaining residual activity appeared to be still further reduced by 10 μM NM-PP1 (FIG. 4B), as expected. Similarly, while NM-PP1 did not affect pT286 for CaMKII wildtype, as expected, the F89G mutant did not demonstrate any significant phosphorylation of T286 at all (FIG. 10A). In HEK cells, GFP-CaMKII F89G did not colocalize with mCh-GluN2B after ionomycin stimulation (FIG. 4C), similar as seen for the kinase-dead K42M mutation that is impaired for binding of nucleotide (FIG. 10B). However, addition of 10 μM NM-PP1 fully enabled binding of CaMKII F89G to GluN2B in HEK cells (FIG. 4C). Similarly, NM-PP1 enabled binding of CaMKII F89G to GluN2B also in vitro (FIG. 10C). Together these results strongly indicate that the CaMKII F89G mutation dramatically reduces nucleotide binding (which inhibits both activity and GluN2B binding) but allows effective occupation of the mutated nucleotide binding pocket with NM-PP1 (which further reduces any residual enzymatic activity, but now enables GluN2B binding). This conclusion also predicts that the LTP stimulus-induced movement of CaMKII to excitatory synapses should be (i) impaired in the CaMKII F89G mutant; but (ii) normalized by pharmacogenetic rescue with NM-PP1. Indeed, both predictions were observed experimentally (FIGS. 4D-4E). Whereas the F89G mutant impaired both basal and LTP-induced synaptic CaMKII localization to the same extent as the K42M mutant that also impairs ATP binding (FIG. 4D), this impaired localization was rescued by NM-PP1 only for the F89G but not the K42M mutant (FIG. 4E), demonstrating specificity and selectivity of the NM-PP1 effect.

### Example 8

### NM-PP1 Restores LTP in CaMKII F89G Slices

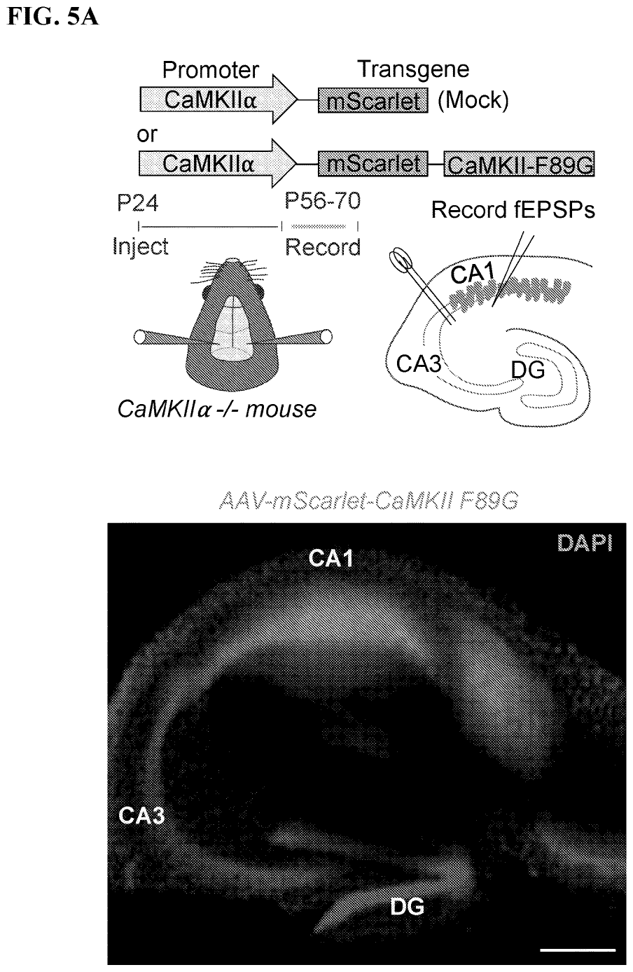

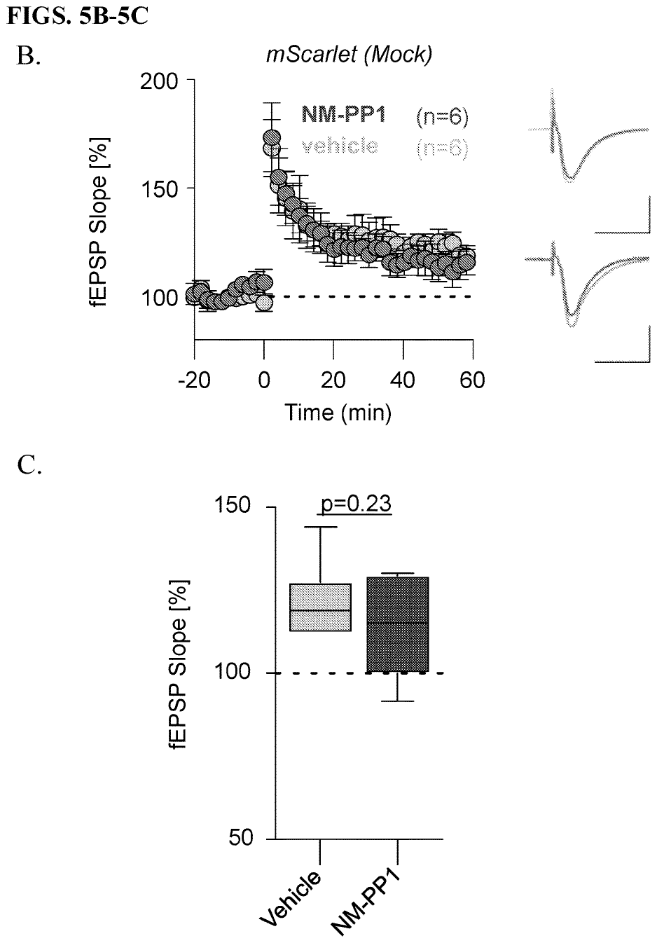

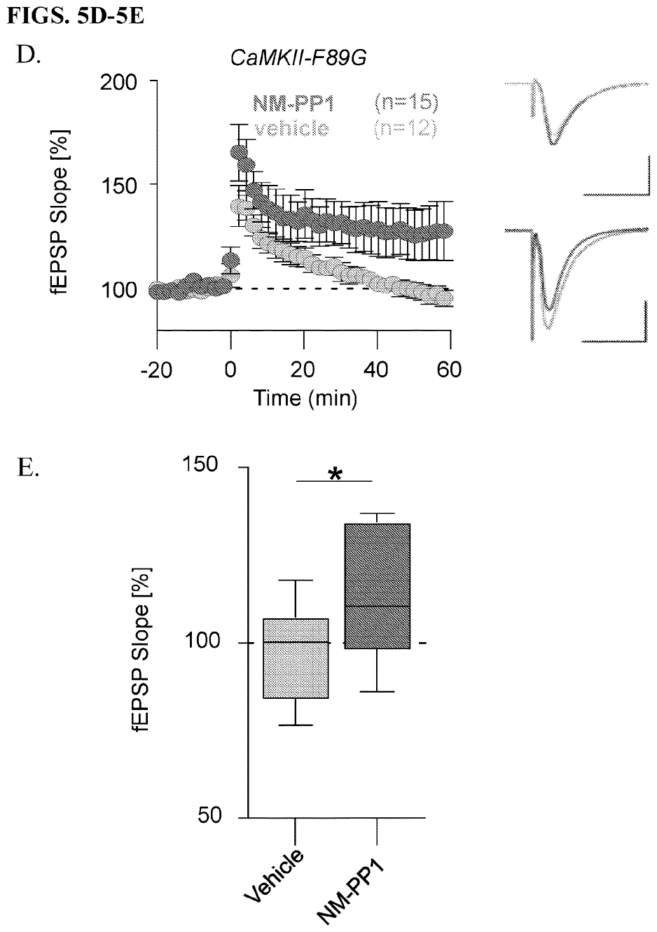

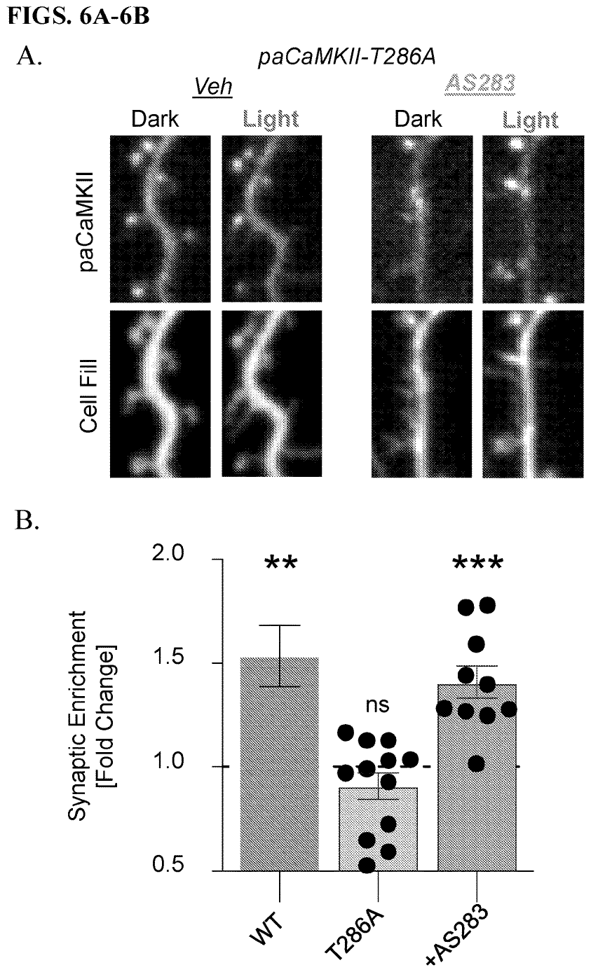

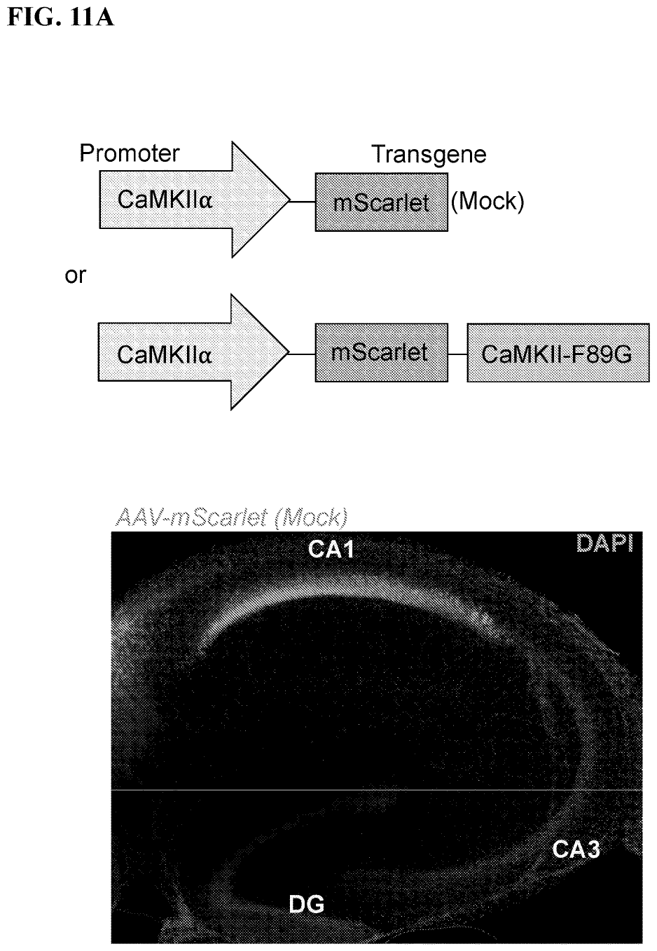

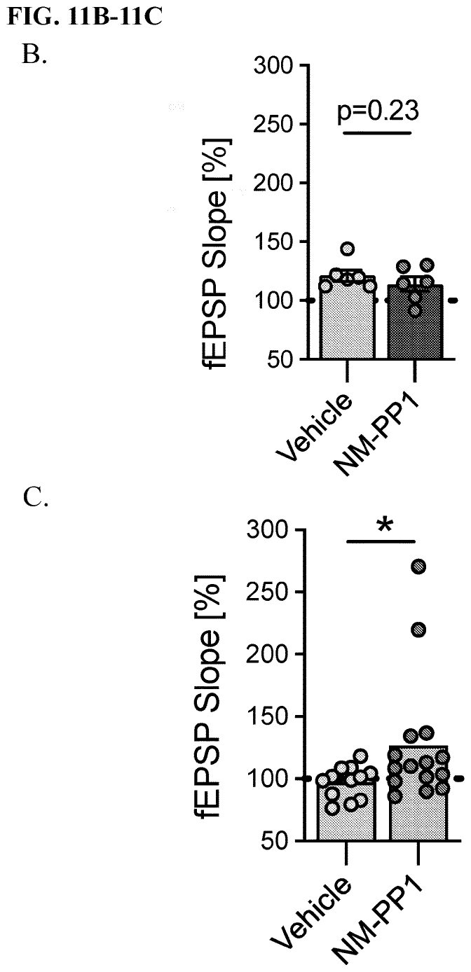

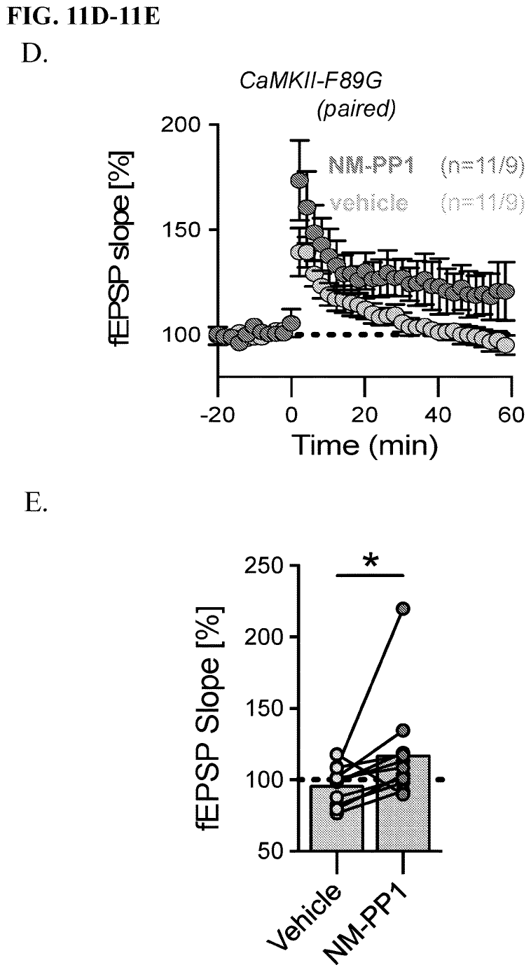

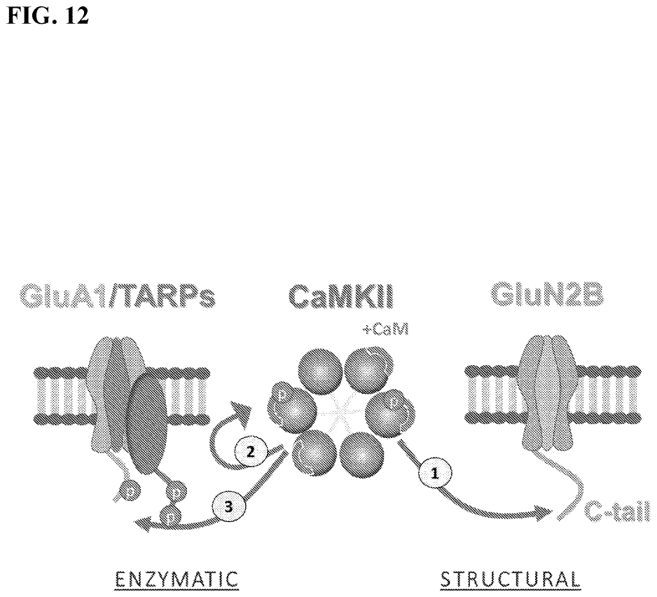

In this Example, further evidence is presented to validate previous results demonstrating that only structural CaMKII functions are needed for LTP induction. Specifically, in one exemplary method, pharmacogenetic rescue of LTP with NM-PP1 was tested in hippocampal slices expressing the CaMKII F89G mutant. For this purpose, mScarlet-labeled CaMKII F89G mutant were re-expressed in the hippocampal CA1 region of CaMKIIα knockout mice. This was done by stereotactic injection of a packaged adeno-associated virus (AAV) construct in which the expression of either mScarlet alone or the mScarlet-labeled mutant CaMKII is controlled by the CaMKIIα promoter (FIG. 5A). After 5 weeks of expression, acute slices were prepared and evaluated for proper localization of expression by imaging of mScarlet fluorescence (FIG. 5A and FIG. 11A). In the CaMKIIα knockout slices that expressed the mScarlet control, LTP was significantly reduced (compare to FIG. 3) but not completely abolished (FIGS. 5B-5C), consistent with previous studies on these knockout mice. As expected, NM-PP1 had no effect on the residual LTP in these control slices (FIGS. 5B-5C and FIG. 11B). By contrast, in slices expressing the CaMKII F89G mutant, LTP was completely abolished (FIGS. 5D-5E), consistent with residual LTP being further suppressed by the dominant negative effect (on the remaining minor CaMKIIβ isoform in the CaMKIIα knockout) that is exerted by a CaMKII mutant with impaired ATP-binding and thereby is deficient in both activity and GluN2B binding. However, most importantly, in these slices, LTP induction was enabled by addition of the NM-PP1 inhibitor (10 μM) (FIGS. 5D-5E and FIGS. 11C-11E) that further reduces enzymatic activity but enables GluN2B binding of the F89G mutant (see FIG. 4). Thus, two independent lines of pharmacogenetic evidence topple current dogma by directly supporting the conclusion that LTP induction is linked to structural rather than enzymatic functions of CaMKII.

### Example 9

CaMKII Binding to GluN2B is Sufficient for sLTP

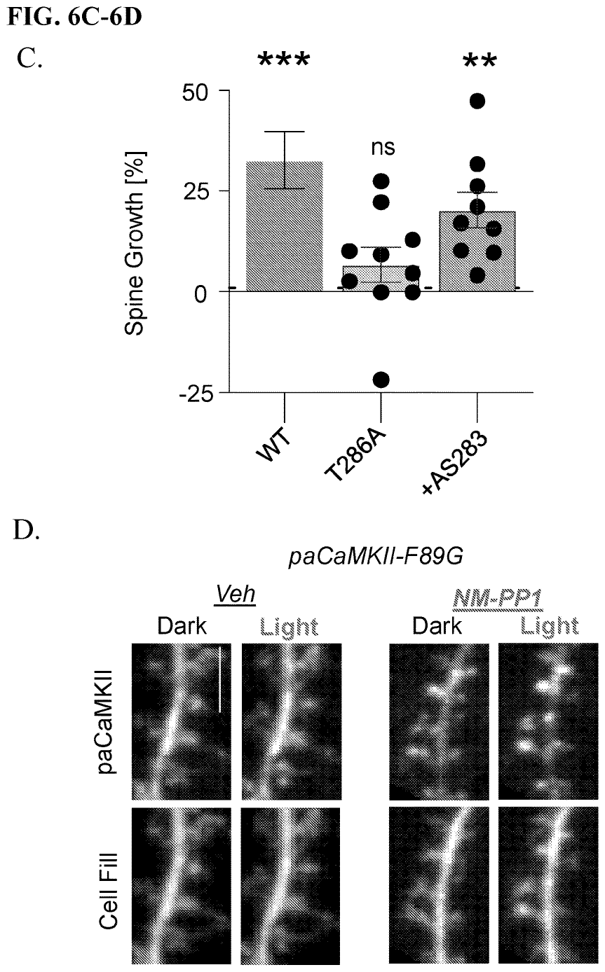

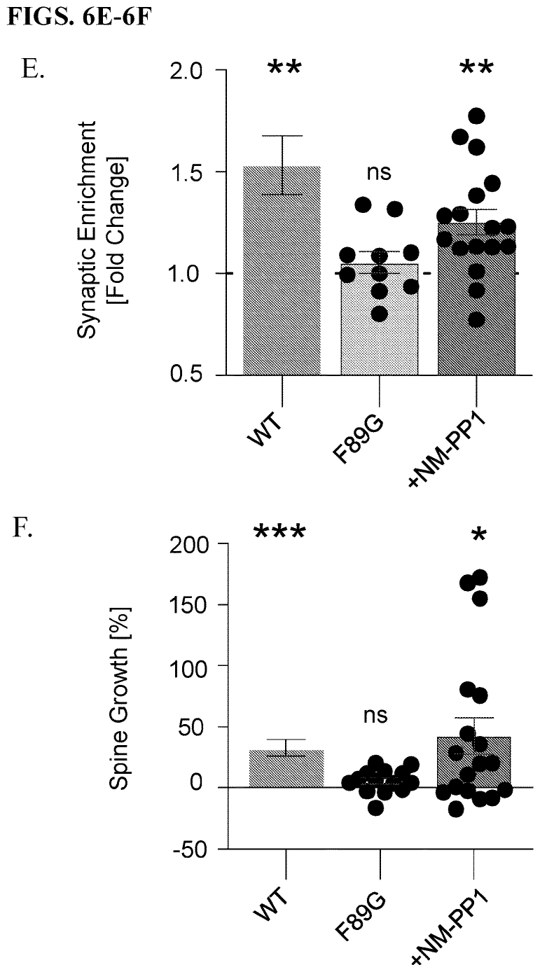

In this Example, two sets of pharmacogenetic tools were used in combination with the photoactivable paCaMKII to test whether structural CaMKII functions are not only the necessary but also the sufficient CaMKII functions in LTP. Specifically, in one exemplary method, hippocampal neurons were examined for sLTP (e.g., the LTP-associated growth of dendritic spines) in response to direct photoactivation of paCaMKII without any other stimulation that could trigger additional signaling pathways. Similar to what was observed with paCaMKII I205K or K42M (see FIG. 1), photoactivation of paCaMKII T286A or F89G did not trigger any significant CaMKII movement to spines or spine growth (FIG. 6A-6C). However, in the presence of the AS283 inhibitor (10 μM), photoactivation of the paCaMKII T286A mutant triggered robust CaMKII movement and spine growth, to a similar extent as seen for paCaMKII wildtype (FIGS. 6A-6C). In a parallel finding, addition of NM-PP1 (10 μM) enabled similar movement and spine growth after photoactivation of paCaMKII F89G (FIGS. 6D-6F). In both cases, suppressing enzymatic CaMKII activity while allowing or enhancing GluN2B binding enabled induction of structural LTP. These results are consistent with the corresponding rescue of CA1 LTP in hippocampal slices (see FIGS. 3C-3D and FIGS. 5D-5E). Additionally, as the photoactivation of paCaMKII does not trigger any other signaling pathways, these results indicate that the structural CaMKII functions are not only necessary but also sufficient CaMKII functions for induction of structural LTP.

### Overview of Examples 1 to 9

Examples 1 to 9, collectively, provide three independent lines of investigation demonstrating that LTP induction is linked to structural CaMKII functions, rather than enzymatic activity towards other substrate proteins (see schematic summary in FIG. 12); the direct photoactivation of CaMKII binding to GluN2B additionally indicated that these structural functions are needed and are also sufficient functions of CaMKII in LTP induction. Importantly, these LTP induction conditions (both electrically and by light) are (i) relatively mild, (ii) expected to engage the main LTP mechanisms that are studied at the hippocampal CA3 to CA1 synapse and (iii) similar to the protocols used previously to suggest involvement of kinase activity. This does not formally rule out that there could be some forms of LTP that may need enzymatic CaMKII activity, but it demonstrates that such activity is not necessary, neither in principle nor in a major form of hippocampal LTP studied. Notably, while phosphorylation of the CaMKII substrate site on another protein, S831 on the AMPA-type glutamate receptor (AMPAR) subunit GluA1, should promote LTP (as it increases single channel conductance), there has been strong previous indication that this phosphorylation is not required for LTP. Other CaMKII phosphorylation sites suggested to promote LTP include S277 and S281 on TARPγ-8, an auxiliary protein that aids in AMPAR trafficking and function. However, another study found that mutating the CaMKII sites on TARPγ-8 did not significantly reduce LTP. In contrast to phosphorylation of the exogenous substrate sites on GluA1 and TARPγ-8, the autophosphorylation of CaMKII at T286 is required for LTP. However, according to these results, the function of pT286 in LTP is not for the generation of the Ca²⁺-independent “autonomous” activity as generally suspected, but instead to enable the needed regulation of synaptic CaMKII accumulation and the coinciding structural CaMKII functions. Thus, generation of pT286 is part of the initial upstream signal processing that leads to LTP induction and enables the downstream output through the structural functions of CaMKII, without any requirement for enzymatic activity towards exogenous substrates. Notably, the fact that LTP can be induced and then maintained in the T286A mutant mice with the help of the AS283 inhibitor (which inhibits enzymatic activity while promoting GluN2B binding) also indicates that LTP maintenance does not require autonomous activity generated by pT286 either. Surprisingly, the complex biochemical regulation of the pT286 reaction (that includes dual need for CaM and cross-regulation with other autophosphorylation reactions) favors its participation in signal processing rather than LTP maintenance. Whereas structural CaMKII functions as disclosed herein are clearly central to LTP induction. Ultimately, any structural LTP mechanism has to regulate the F-actin cytoskeleton. However, while direct F-actin binding and bundling is among the structural functions of CaMKII, this is largely restricted to the CaMKIIβ isoform rather than the major a isoform studied here. Moreover, binding to F-actin versus GluN2B is oppositely regulated, with activating signals disrupting the F-actin binding rather than inducing it. These findings support use of the inhibitors disclosed herein in therapeutic applications for chronic CaMKII inhibition in the treatment of various conditions.

### Example 10

ATP Competitive CaMKII Inhibitors Restores cLTP Blocked by Soluble Amyloid β

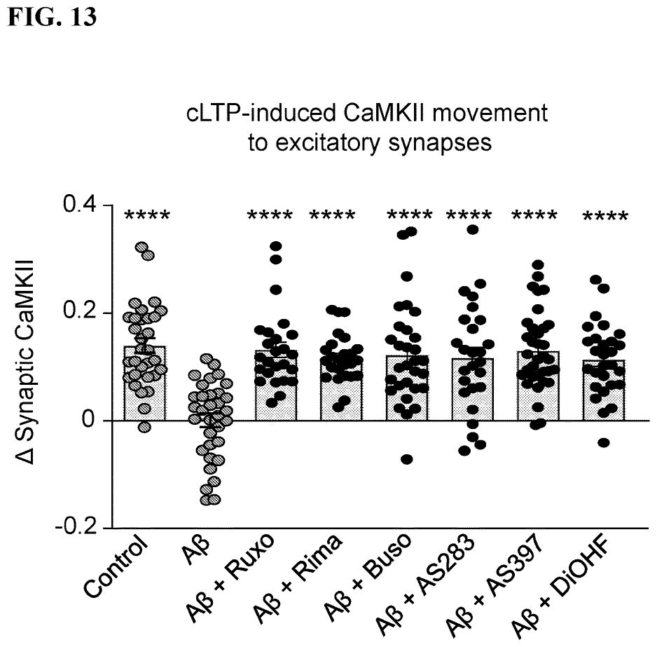

In an exemplary method, the effect of soluble oligomeric amyloid β peptide (Aβ) on migration of CaMKII to excitatory synapses after chemical LTP stimulation was tested. Specifically, hippocampal slices transfected to express mCh-PSD-95 intrabody, iRFP cell fill, and GFP-CaMKII were incubated with 0.5 μM Aβ or vehicle, together with various ATP-competitive CaMKII inhibitors at 10 μM: JAK inhibitors were used; ruxolitinib (ruxo); rimacalib (rima; also termed SMP-114); bosutinib (bosu); and other CaMKII ATPase inhibitors, AS100283 (AS283); AS100397 (AS397); or 3′,4′-dihydroflavonol (DiOHF; also termed NP202) for 45 minutes prior to cLTP stimulation (100 μM glutamate and 10 M glycine for 45 seconds). It was found that Aβ blocks cLTP induced movement of CaMKII to the synapses and this could be rescued by each ATP competitive CaMKII inhibitors tested (FIG. 13). Thus, ATP competitive CaMKII inhibitors can reverse the loss of LTP attributed to soluble Aβ.

### Example 11

### AS397 Restores Structural LTP Blocked by Phosphorylation Incompetent CaMKIIα (CaMKII T286A)

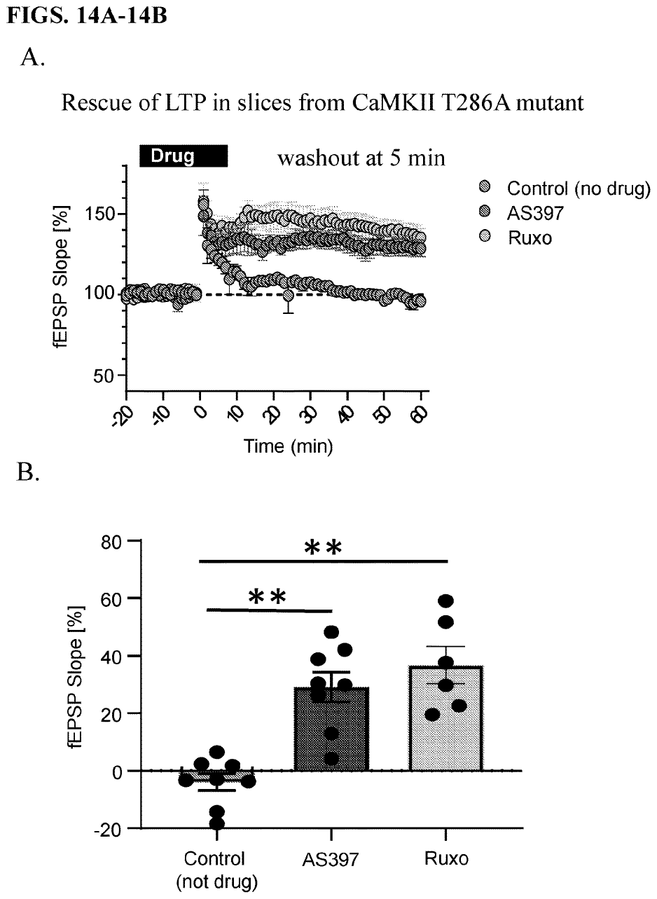

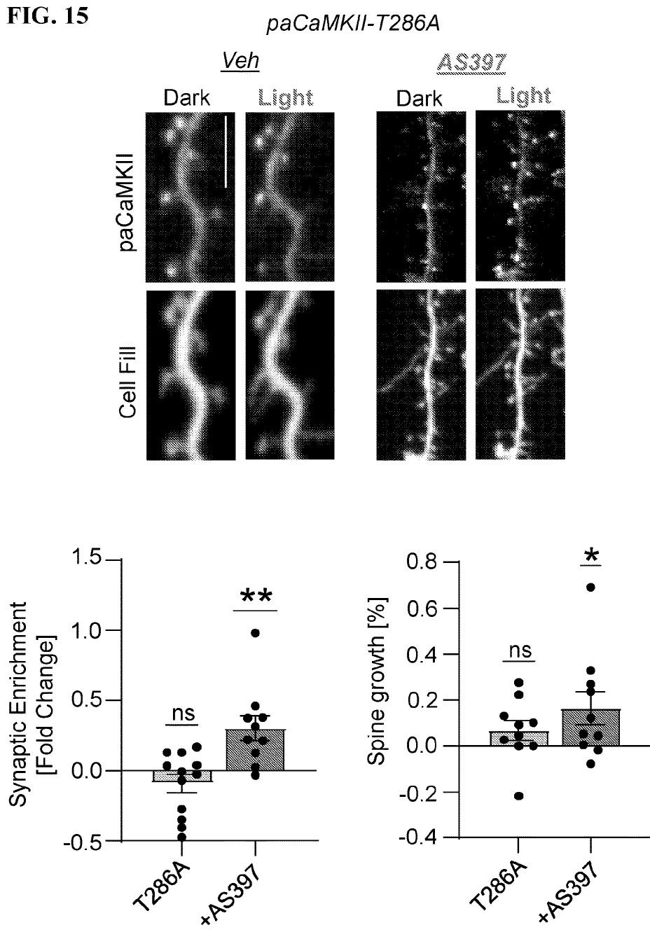

In this example, the effect of ATP-competitive CaMKII inhibitors, AS100397 (AS397) and/or ruxolitinib (ruxo) on LTP induction was tested. In a first exemplary method, LTP was induced in hippocampal slices from CaMKII T286A mutant mice in the presence or absence of AS397 or ruxo at 10 μM. As illustrated in FIGS. 14A-14B, LTP was blocked by the T286A mutation but was restored in the presence of either ATP competitive CaMKII inhibitor, similar to what was observed with AS283 (e.g., See FIG. 3A-3B). In another exemplary method, a photoactivatable paCaMKII with a T286A mutation was used, as described in Example 1, to track structural LTP-visualized as the localization (or lack thereof) of paCaMKII to synapses after light stimulation. It was found that, like AS283 (e.g., See FIG. 6A-6C), the ATP-competitive CaMKII inhibitor AS397 restores light-induced movement of the photoactivatable mutant paCaMKII (T286A) to excitatory synapses in hippocampal neurons and restored the structural LTP measure by spine growth (FIG. 15).

### Example 12

AS397 and Ruxolitinib do not Interfere with GluN2B Binding In Vitro or within Cells

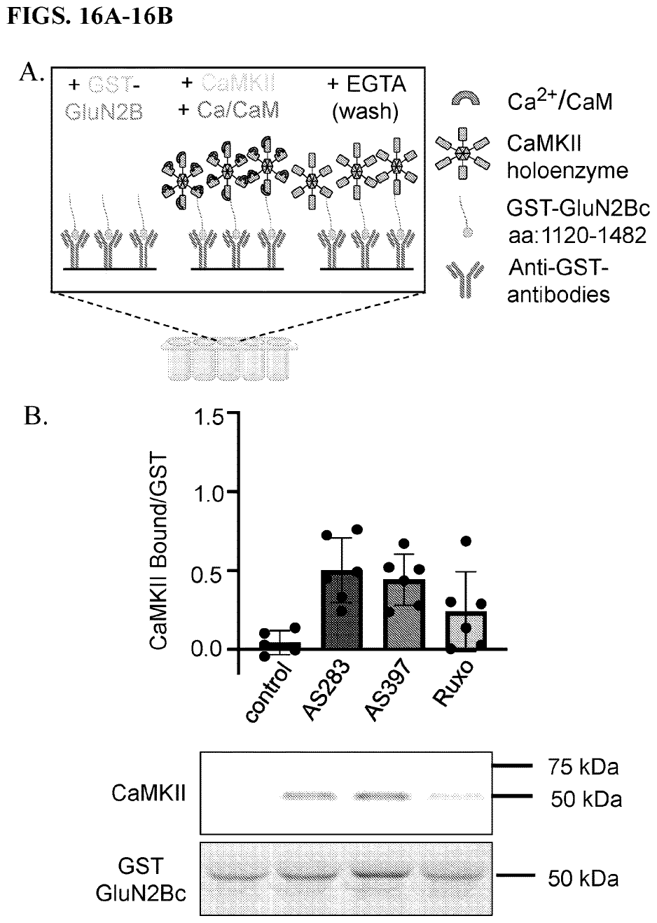

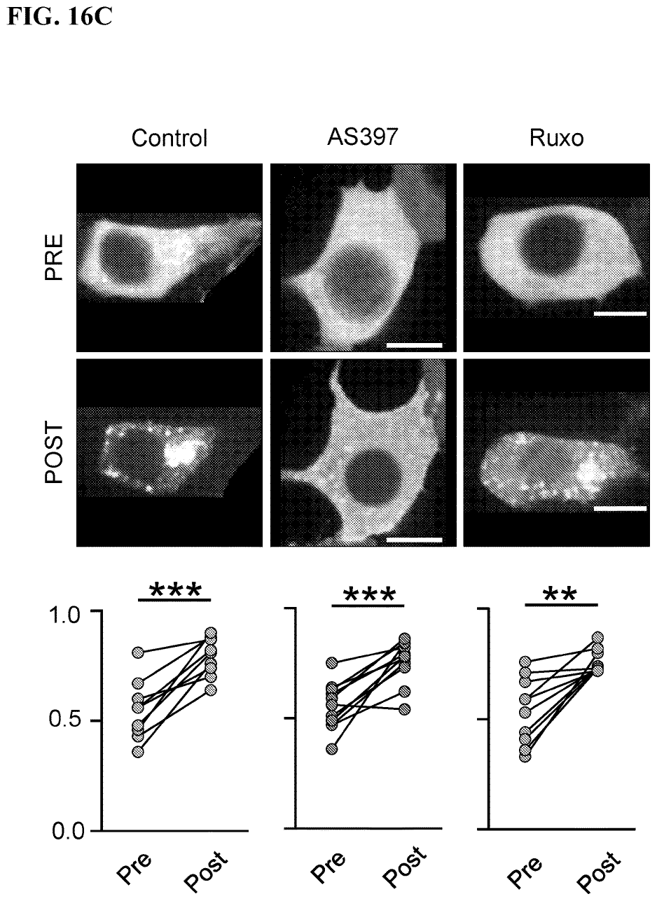

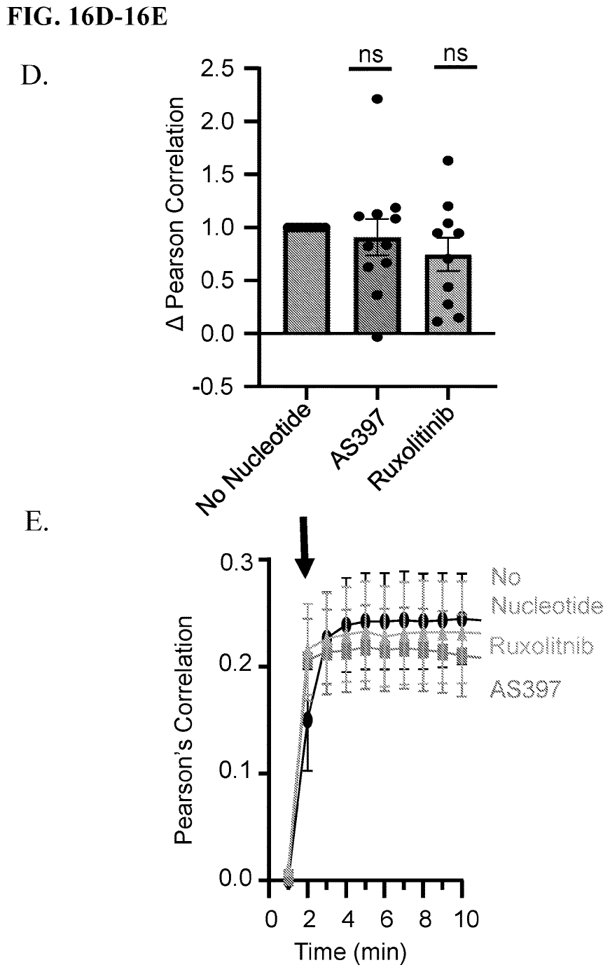

In another exemplary method, the ability of ATP competitive CaMKII inhibitors to interfere with CaMKII binding to GluN2B in vitro or within cells was tested. Briefly, as illustrated in FIG. 16A, GST-GluN2B-c was immobilized on anti-GST plates. CaMKII, Ca²⁺/CaM, reaction mix, and nucleotide or ATP-competitive inhibitor were added (10 μM AS397, AS283 and Ruxolitinib), followed by an EGTA wash. Binding of CaMKII to the immobilized GluN2B was examined immunoblot. FIG. 16B represent illustrative immunoblots and quantification demonstrating that each ATP-competitive inhibitor increased CaMKII binding compared to a control without nucleotide or inhibitor. In another exemplary method, co-localization of GFP-CaMKII (Green) with a membrane targeted mCherry-GluN2B C-tail (red) in HEK293 cells was measured after ionomycin stimulation (as described in earlier Examples). These cellular CaMKII/GluNTB binding assays demonstrated again that AS397 and Ruxolitinib (applied for 15 minutes at 10 μM before ionomycin stimulation) did not affect ionomycin induced CaMKII/GluNTB binding in cells (FIG. 16C) and mirror results demonstrated earlier for AS293 (see e.g., FIG. 2D). FIG. 16D further quantify the results visualized in FIG. 16C and illustrate that the net change in CaMKII/GluN2B co-localization after drug incubation and ionomycin treatment was not significant compared to controls. Likewise, the time-course of CaMKII co-localization with GluN2B in these cells after ionomycin treatment was not affected by the presence of the inhibitors (FIG. 16E). These experiments again demonstrate that ATP competitive inhibitors like AS397 and Ruxolitinib do not interfere with GluN2B and CaMKII binding in vitro or in cells.

### Example 13

The ATP-Competitive CaMKII Inhibitor AS100397 (AS397) does not Disrupt LTP Maintenance

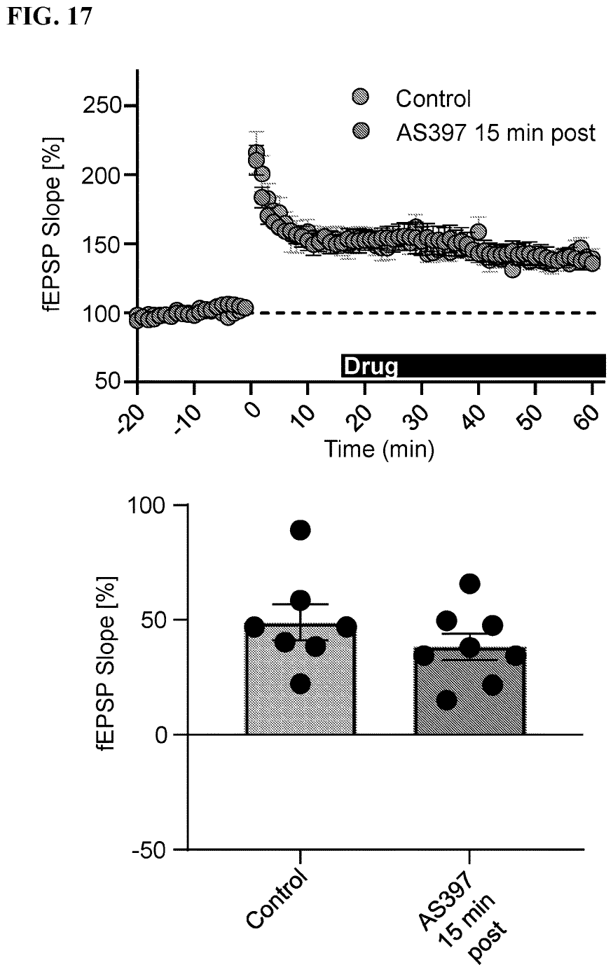

In another exemplary method, whether an ATP-competitive CaMKII inhibitor might interfere with LTP maintenance (rather than induction) was tested. As demonstrated in earlier examples (e.g., in FIG. 3A-3B), an ATP-competitive inhibitor AS283 does not disrupt LTP induction in WT slices when added 15 minutes before LTP induction and washed out 5 minutes after LTP induction. To test whether these compounds interfere with LTP maintenance then, an exemplary method was performed where the CaMKII inhibitor (AS397) was applied starting 15 minutes after LTP induction and maintained for the duration of the time LTP was measured. It was found that the presence of this ATP-competitive CaMKII inhibitor (AS397) did not affect the maintenance of LTP (FIG. 17). Therefore, these observations support that any possible LTP impairment by ATP-competitive CaMKII inhibitors would be limited to a narrow window of about 5-15 minutes after LTP induction. This supports the observation that these agents can be applied as chronic inhibitors of adverse CaMKII activities; for example, when taken during times when little to no acute learning is required.

### Materials and Methods

Experimental animals: All animals were housed in ventilated cages on a 12 h light/12 h dark cycle and were provided ad libitum access to food and water. Male wildtype and T286A KI mice (on a C57BL/6 background) from heterozygous breeder pairs (8-12 weeks old) were used for slice electrophysiology. Mixed sex CaMKII KO mice were used for AAV-injections and slice electrophysiology. Mixed sex pups from Sprague-Dawley rats (P0, Charles River) or individual pups from heterozygous breeding of the GluN2B^{ΔCaMKII} mutant mice were used to prepare dissociated hippocampal cultures for imaging. The mutant mice used here were described previously: the CaMKIIα knockout line used here was made; the GluN2B^{ΔCaMKII} line was provided; and the T286A line was provided.

Material and DNA constructs: Material was obtained from Sigma, unless noted otherwise. CMV-mEGFP (A206K)-paCaMKII was a gift from Hideji Murakoshi (Addgene plasmid #165438). The pAAV-CaMKIIα-mScarlet vector was a gift (Addgene #131000). CaMKIIα-F89G cDNA was cloned into to MCS after mScarlet using BsrGI & EcoRV.

Protein purification: Expression and purification of CaMKII, CaM, GST-GluN2Bc and GST-GluA1 were conducted according to established protocol described in detail previously. CaMKIIα was purified from a baculovirus/Sf9 cell expression system. CaM and GST-GluN2Bc WT and mutant constructs were purified from BL21 bacteria.

Immunoblot analysis. Protein concentration was determined using the Pierce BCA protein assay (Thermo-Fisher). Before undergoing SDS-PAGE, samples were boiled in Laemmli sample buffer for 5 min at 95° C. Proteins were separated in a resolving phase polymerized from 10% acrylamide, then transferred to a polyvinylidene difluoride membrane at 24 V for 1-2 h at 4° C. Membranes were blocked in 5% milk or BSA and incubated with anti-CaMKIIα (1:4000, CBα2, available at Invitrogen but made in house), anti-CaMKIIα (1:2000, BD) pT286-CaMKII (1:2500, Phospho-Solutions), anti-GST (1:2000, Millipore), pS831-GluA1 (1:2000, Phospho-Solutions), pS1303 (1:2000, Millipore) followed by either Amersham ECL goat anti-mouse or anti-rabbit HRP-linked secondary 1:10000 (GE Healthcare). Blots were developed using chemiluminescence (Super Signal West Femto, Thermo-Fisher) imaged using the Chemi-Imager 4400 system (Alpha-Innotech), or imaged directly by fluorescence (Cytiva CyDye 700 goat anti-mouse and CyDye 800 goat anti-rabbit secondary antibodies.) using an OdysseyFc imaging instrument. All immunoblots were analyzed by densitometry (ImageJ). Phospho-signal was corrected to total protein. Relative band intensity was normalized as a percent of control conditions on the same blot, which was set at a value of one to allow for comparison between multiple experiments.

In vitro phosphorylation assays. CaMKII-mediated phosphorylation of GluA1 S831 was measured by in vitro kinase reaction with purified GST-GluA1 c-terminal tail. Reactions contained 40 nM CaMKII (or water for negative controls), 1 μM GST-GluA1, 50 mM PIPES pH 7.1, 2 mM CaCl₂), 10 mM MgCl₂, 1 μM calmodulin, 1-4 mM ATP, and 1 PM of okadaic acid. Reactions were done at 30° C. for 20 s; reactions were stopped by adding SDS-loading buffer and incubation in a boiling water bath for 5 min. GST, phospho-S831, CaMKII, phospho-T286, or phospho-S1303 were detected in the samples by immunoblot analysis.

Km and Ki determination. The inhibition constant Ki was calculated using the Cheng-Prusoff equation for competitive inhibition: Ki=IC50/(1+[ATP]/Kₘ). Using Michaelis-Menten analyses the CaMKII Kₘ for ATP at the described experimental conditions was experimentally determined to be KₘATP=33.3 μM (FIG. 8B). This is within the range of previously published Kₘ values (8-127 μM).

CaMKII binding to GluN2B in vitro. CaMKII/GluN2B binding assays were done as described. Briefly, GST-GluN2B-c-tail (GST-2BC) was immobilized on anti-GST-antibody-coated microtiter plates (Thermo Scientific), blocked for 30 min with 5% BSA, and then overlaid with 40 nm CaMKII (subunit concentration) in PIPES-buffered saline (pH 7.2) containing: 2 mM Ca²⁺, 1 μM CaM, 1 mM Mg²⁺, 0.1% BSA, 0.1% Tween-20 for 20 min at room temperature. Addition of 1 mM ADP, 1 mM ATP, 10 μM AS283 (or AS105) or 10 AM NM-PP1 was added accordingly (see figure legends). After extensive washes in buffer containing 1 mM EGTA, GST-2BC and bound CaMKII was eluted for 10 min in SDS-loading buffer at 95° C. Bound CaMKII was measured via immunoblot.

Live imaging of HEK cells. HEK cells were grown and transfected with expression vectors for GFP-CaMKII mutants and pDisplay-mCh-GluN2B-c tail (2BC) as previously described; the GluN2B construct used included a S1303A mutation to avoid potential complications by differential phosphorylation of this regulatory site in the different conditions with or without kinase inhibitors. GFP-CaMKII colocalization with GluN2B in response to a Ca²⁺ stimulus induced by 10 μM ionomycin was monitored for 5-10 min at 32° C. in imaging buffer (0.87× Hanks Balanced Salt Solution, 25 mM Hepes pH 7.4, 2 mM glucose, 2 mM CaCl₂), 1 mM MgCl₂) by fluorescence microscopy. Images were acquired on a Zeiss Axiovert 200M equipped with a climate control chamber, using SlideBook software (Intelligent Imaging Innovations). Colocalization analysis was performed by calculating the Pearson's correlation (correlation index) of pDisplay-mCh-2BC and GFP-CaMKII within the cytoplasm of HEK cells after background subtraction.

paCaMKII stimulation in HEK cells. HEK cells were transfected with GFP-paCaMKII (WT, K42M, I205K) and pDisplay-mCherry-2BC$1303A and left in the dark for 10 minutes prior to photoactivation to ensure that paCaMKII was in the dark state. GFP-paCaMKII was then photoactivated and imaged simultaneously via confocal imaging over a 3 μm Z-stack (step size: 0.6 μm) or a single plane with 488 nm excitation once per minute for a total of 5 minutes. Correlation index was measured the same as for ionomycin-induced colocalization.

Primary hippocampal culture preparation. To prepare primary rat hippocampal neurons, hippocampi were dissected from mixed sex rat pups (P0), dissociated in papain for 1 h, and plated at 100,000 cells/well on glass coverslips in 12-well culture dishes for imaging, and 500,000 cells/well on 6-well culture dishes for biochemistry. Mouse hippocampi were dissected from individual mouse pups (P1-2), dissociated in papain for 30 m, and plated at 200,000-250,000 cells/mL on glass coverslips for imaging. At DIV 14-18, neurons were transfected with 1 μg total cDNA per well using Lipofectamine 2000 (Invitrogen), then imaged 2-3 days later.

Chemical LTP stimulation. Chemical LTP (CL TP) was induced with 100 μM glutamate and 10 μM glycine for 45 seconds. Treatments were followed by washout in 5 volumes of fresh ACSF. For biochemistry experiments, neurons were treated with 1 μM TTX to silence neurons prior to treatment with cLTP.

paCaMKII stimulation in hippocampal neurons. Neurons were wrapped in aluminum foil immediately following transfection and only exposed to red light to imaging. One image was then taken of each neuron to serve as a pre-photoactivation baseline. Immediately following this baseline image, paCaMKII was globally photoactivated with 405 nm laser pulse (100 ms exposure, 75% laser power) once every 10 seconds, for a total of 60 seconds. Neurons were then imaged 15 minutes after stimulation and assessed for CaMKII synaptic enrichment and dendritic spine growth.

Image analysis of hippocampal neurons. DIV 15-18 neuronal cultures were transfected to express mCh-PSD-95 intrabody, iRFP cell fill, and GFP-CaMKII (WT or noted mutations) and imaged 24-48 h later. All experiments with overexpression of CaMKII mutants was co-transfected with shRNA for CaMKII 5′UTR to knock down endogenous CaMKII. Images were collected at 32° C. in HEPES buffered imaging solution containing (in mM) 130 NaCl, 5 KCl, 10 HEPES pH 7.4, 20 Glucose, 2 CaCl₂), 1 MgCl₂. Images of individual neurons from two independent cultures were acquired by 0.5 μm steps over 6 μm. 2D maximum intensity projection images were then generated and analyzed using a custom-build program in ImageJ. The program utilizes combinatorial thresholding to mask regions of the cell that contain high intensity PSD-95 puncta (the post-synaptic side of excitatory synapses in dendritic spines) and regions of the dendritic shaft that contain no fluorescence intensity of PSD-95. As a measure of synaptic enrichment, the ratio of mean CaMKII fluorescence intensity of the PSD-95 mask to the mean CaMKII fluorescence intensity in the dendritic shaft mask is measured. Spine growth was assessed by measuring the changes in mCherry cell fill fluorescence intensity within dendritic spine ROIs (F/F0).

AAV Production. AAV vectors were constructed from an empty AAV transfer plasmid where the expression cassette is as follows: left-ITR, CaMKIIα promoter, mScarlet, multiple cloning site, WPRE and right ITR. To generate AAVs, HEK293T cells were transfected with a AAV transfer plasmid, pHelper and pRC-DJ. AAVs were purified as previously described. Briefly, 72-hour post transfection, cells were harvested, lysed and virus was purified on an iodixanol gradient via ultracentrifugation. Virus was harvested from the 40% fraction, then concentrated and washed in a 100K MWCO Amicon filter. AAVs were titered by infecting mouse hippocampal cultures with serial dilutions and used for stereotactic infections at 1×10\hat{ }9 infections units/uL. The following AAVs were used: AAV_{DJ}-CaMKIIα-mScarlet-CaMKII^{F89G}, AAV_{DJ}-CaMKIIα-mScarlet.

Stereotactic surgeries. Stereotactic injections were performed on P24 CaMKIIα-KO mice. Animals were anesthetized with an intraperitoneal injection of 2,2,2-Tribromoethanol (250 mg/kg) then head fixed to a stereotactic frame (KOPF). AAVs (0.2-0.5 μL) were injected into intermediate CA1 at a rate of 10 mL/hr using a syringe pump (World Precision Instruments). Coordinates (in mm): anterior-posterior: −3.17, mediolateral: ±3.45 (relative to Bregma), and dorsoventral: −2.5 (relative to pia). To confirm specificity of the injection site following LTP recordings, slices were fixed in 4% PFA, then cover slipped and imaged on an Olympus slide scanning microscope.

Hippocampal slice preparation. WT and mutant mouse hippocampal slices were prepared using P56-70 mice. Isoflurane anesthetized mice were rapidly decapitated, and the brain was dissected in ice-cold high sucrose solution containing 220 mM sucrose, 12 mM MgSO₄, 10 mM glucose, 0.2 mM CaCl₂), 0.5 mM KCl, 0.65 mM NaH₂PO₄, 13 mM

NaHCO₃, and 1.8 mM ascorbate. Transverse hippocampal slices (400 μm) were made using a tissue chopper (Mcllwain) and transferred into 32° C. artificial cerebral spinal fluid (ACSF) containing 124 mM NaCl, 3.5 mM KCl, 1.3 mM NaH₂PO₄, 26 mM NaHCO₃, 10 mM glucose, 2 mM CaCl₂), 1 mM MgSO₄, and 1.8 mM ascorbate. All slices were recovered in 95% O₂/5% CO₂ for at least 1.5 hours before experimentation.

Extracellular field recordings. For electrophysiological slice recording experiments, a glass micropipette (typical resistance 0.4 to 0.8 megaohm when filled with ACSF) was used to record field excitatory postsynaptic potentials (fEPSPs) from the CA1 dendritic layer in response to stimulation of the Schaffer collaterals using a tungsten bipolar electrode. Slices were continually perfused with 30.5°+0.5° C. ACSF at a rate of 2.5±0.5 ml/min during recordings. Stimuli were delivered every 20 s, and three responses (1 min) were averaged for analysis. Data were analyzed using WinLTP software with slope calculated as the initial rise from 10 to 60% of response peak. Input/output (I/O) curves were generated by increasing the stimulus intensity at a constant interval until a maximum response or population spike was noted to determine stimulation that elicits 50% of maximum slope. Paired-pulse recordings (50-ms interpulse interval) were acquired from 50% max slope, and no differences in presynaptic facilitation were seen in mutant slices. A stable baseline was acquired for a minimum of 20 min at 50% maximum slope before high frequency stimulation (HFS; 2×100 Hz. 10 sec interval) was applied. Responses were recorded for 60 min after HFS. Change in slope was calculated as a ratio of the average slope of the 20 min baseline (before HFS). Bar graphs of % fEPSP slope were calculated by averaging the 50-60-minute timepoints post HFS and normalized to baseline.

Soluble Aβ preparation. Aβ was prepared as follows: Amyloid beta (1-42) peptide was purchased from Anaspec. Upon arrival, the peptide was resuspended in 1,1,1,3,3,3-hexafluoro-2-propanol (HFP) and aliquoted in separate tubes. The solubilized peptide was left overnight at room temperature and then stored at −80° C. To reconstitute Aβ for experiments, 6 mL DMSO is added to the lyophilized Aβ tube and vortexed vigorously. An additional 54 mL of PBS was added to the tube and vortexed again to achieve a concentration of 100 mM. Then, the 60 mL of DMSO/PBS/Ab was left at 4° C. overnight or for two nights and then centrifuged at 14,000 rpm for 10 min. The supernatant was diluted as needed in PBS and used for experiments.

Statistical Analysis. All data are shown as mean±SEM. Statistical significance is indicated in the figure legends. Statistics were performed using Prism (GraphPad) software. Imaging experiments were obtained using SlideBook 6.0 software and analyzed using ImageJ. Immunoblots were analyzed by densitometry using ImageJ (NIH). All data were tested for their ability to meet parametric conditions, as evaluated by a Shapiro-Wilk test for normal distribution and a Brown-Forsythe test (3 or more groups) or an F-test (2 groups) to determine equal variance. All comparisons between two groups met parametric criteria, and independent samples were analyzed using unpaired student's t-tests. Comparisons between three or more groups meeting parametric criteria were done by one or two-way ANOVA with specific post-hoc analysis indicated in figure legends.

1. A method for preventing, treating, ameliorating, or eliminating a health condition in a subject comprising:
administering to the subject a composition comprising one or more ATP-competitive CaMKII inhibitors, wherein the composition preserves CaMKII structural function while preventing, treating, ameliorating, or eliminating a health condition in a subject.

2. The method according to claim 1, wherein the health condition comprises a health condition affecting the brain of the subject.

3. The method according to claim 1, wherein the health condition comprises a health condition affecting one or more of learning, memory, and cognition in the subject.

4. The method according to claim 2, wherein the brain condition is a side effect of a health condition, and the health condition comprises one or more of Down Syndrome (DS), Alzheimer's Disease (AD), a cerebral ischemia, a traumatic brain injury (TBI), other brain condition or brain injury, or a combination thereof.

5. The method according to claim 1, wherein the health condition comprises a neurological condition due to adverse activities of Aβ precursor protein (APP), oligomeric amyloid-β peptide (Aβ), or combination thereof.

6. The method according to claim 1, wherein the one or more ATP-competitive CaMKII inhibitors comprises one or more of AS100105, AS100283, AS100397, bosutinib, sunitinib, RA306, RA608, GS-680, Scios-15b, diaippon, dainipponB:25, 3′,4′-dihydroflavonol (DiOHF), NP202), rimacalib/smp-114, ruxolitinib, barictinib, other Janus Kinase (JAK) inhibitors, other ATP CaMKII inhibitors, or a combination thereof.

7. The method according to claim 1, wherein the one or more ATP-competitive CaMKII inhibitors comprise AS100283, AS100397, ruxolitinib, barictinib or a combination thereof.

8. The method according to claim 1, further comprising administering a standard treatment for improving learning, memory, and cognition in the subject at least one of: in combination with, before, during, or after administering the one or more ATP-competitive CaMKII inhibitors to the subject.

9. The method according to claim 1, wherein the one or more ATP-competitive CaMKII inhibitors or JAK inhibitors do not reduce or preserve long-term potentiation (LTP) mechanisms in the subject; optionally, wherein the one or more ATP-competitive CaMKII inhibitors preserve CaMKII movement to synapses and preserves CaMKII synaptic activity.

10. The method according to claim 1, wherein the one or more ATP-competitive CaMKII inhibitors restore LTP mechanisms impaired by β-amyloid peptide (Aβ) in a subject compared to a subject having impaired LTP mechanisms due to β-amyloid peptide (Aβ) not treated with the one or more ATP-competitive CaMKII inhibitors.

11. The method according to claim 1, wherein the one or more ATP-competitive CaMKII inhibitors do not inhibit CaMKII binding to N-methyl-D-aspartate (NMDA)-type glutamate receptor (NMDAR) subunit GluN2B

12. A method for preventing, treating, ameliorating, or eliminating a health condition in a subject comprising:
administering to the subject a composition comprising one or more ATP-competitive CaMKII inhibitors, alone or in combination with at least one additional agent wherein the one or more CaMKII inhibitors cross the blood-brain barrier, do not inhibit, or prevent induction of long-term potentiation (LTP) and do not prevent CaMKII inhibition in the brain of the subject while preventing, treating, ameliorating, or eliminating the health condition in the subject.

13. The method according to claim 12, wherein the health condition comprises a condition or trauma that interferes with, or reduces learning, memory and/or cognitive function in the subject.

14. The method according to claim 12, wherein the composition or a combination composition does not reduce learning, memory, or cognitive functions in the subject.

15. The method according to claim 12, wherein administering the composition or combination composition comprises administering the composition or combination composition one time, for a predetermined period, continuously, for long-term treatment of the subject or for the remainder of the life of the subject.

16. The method according to claim 12, wherein administering the composition or combination composition comprises administering the composition or combination composition for long term or for a continuous treatment period three times daily, two times daily, daily, every other day, twice weekly, weekly, bi-monthly, or monthly.

17. The method according to claim 12, wherein the at least one additional agent comprises an anti-β-amyloid peptide agent or other anti-amyloid agent treatment; optionally wherein the agent comprises omega-3 fatty acids, guanidine salts, dimethyl sulfoxide (DMSO), vitamin C, lecanemab, donanemab, or a combination thereof.

18. A composition for preserving or restoring LTP function in a subject, comprising one or more ATP-competitive CaMKII inhibitors, and at least one anti-β-amyloid peptide agent or other anti-amyloid agent.

19. The composition according to claim 18, wherein the one or more ATP-competitive CaMKII inhibitors comprises one or more of AS100105, AS100283, AS100397, bosutinib, sunitinib, RA306, RA608, GS-680, Scios-15b, diaippon, dainipponB:25, 3′,4′-dihydroflavonol (DiOHF), NP202), rimacalib/smp-114, ruxolitinib, barictinib, other Janus Kinase (JAK) inhibitors, other ATP CaMKII inhibitors, or a combination thereof.

20. (canceled)

21. A kit comprising the composition according to claim 18, and at least one container.
